SEQUENCE LISTING (17 sequences)

SEQ ID NO: 1 - DNA, 28 bp, synthetic construct
  source 1..28: mol_type = other DNA
aatgatacggcgaccaccgagactacac

SEQ ID NO: 2 - DNA, 23 bp, synthetic construct
  source 1..23: mol_type = other DNA
caagcagaagacggcatacgaat

SEQ ID NO: 3 - DNA, 23 bp, synthetic construct
  source 1..23: mol_type = other DNA
caagcagaagacggcatacagat

SEQ ID NO: 4 - DNA, 35 bp, synthetic construct
  source 1..35: mol_type = other DNA
gctggcacgtccgaacgcttcgttaatccgttgag

SEQ ID NO: 5 - DNA, 35 bp, synthetic construct
  source 1..35: mol_type = other DNA
ctcaacggattaacgaagcgttcggacgtgccagc

SEQ ID NO: 6 - DNA, 35 bp, synthetic construct
  source 1..35: mol_type = other DNA
cgtcgtctgccatggcgcttcggtggatatgaact

SEQ ID NO: 7 - DNA, 35 bp, synthetic construct
  source 1..35: mol_type = other DNA
agttcatatccaccgaagcgccatggcagacgacg

SEQ ID NO: 8 - DNA, 35 bp, synthetic construct
  source 1..35: mol_type = other DNA
acggccgctaatatcaacgcgtcgaatccgcaact

SEQ ID NO: 9 - DNA, 35 bp, synthetic construct
  source 1..35: mol_type = other DNA
agttgcggattcgacgcgttgatattagcggccgt

SEQ ID NO: 10 - DNA, 34 bp, synthetic construct
  source 1..34: mol_type = other DNA
gccgcgttacgttagccggactattcgatgcagc

SEQ ID NO: 11 - DNA, 34 bp, synthetic construct
  source 1..34: mol_type = other DNA
gctgcatcgaatagtccggctaacgtaacgcggc

SEQ ID NO: 12 - DNA, 29 bp, synthetic construct
  modified_base 23: mod_base = OTHER; note = uracil
  source 1..29: mol_type = other DNA
aatgatacggcgaccaccgagatctacac

SEQ ID NO: 13 - DNA, 29 bp, synthetic construct
  source 1..29: mol_type = other DNA
  modified_base 23: mod_base = OTHER; note = allyl T
aatgatacggcgaccaccgaganctacac

SEQ ID NO: 14 - DNA, 24 bp, synthetic construct
  modified_base 22: mod_base = OTHER; note = uracil
  source 1..24: mol_type = other DNA
caagcagaagacggcatacgatat

SEQ ID NO: 15 - DNA, 24 bp, synthetic construct
  source 1..24: mol_type = other DNA
  modified_base 22: mod_base = OTHER; note = 8-oxoguanine
caagcagaagacggcatacganat

SEQ ID NO: 16 - DNA, 24 bp, synthetic construct
  modified_base 20: mod_base = OTHER; note = 8-oxoguanine
  source 1..24: mol_type = other DNA
caagcagaagacggcatacnagat

SEQ ID NO: 17 - DNA, 24 bp, synthetic construct
  source 1..24: mol_type = other DNA
  modified_base 20: mod_base = OTHER; note = 8-oxoguanine
  modified_base 22: mod_base = OTHER; note = 8-oxoguanine
caagcagaagacggcatacnanat

(19) United States
(12) Patent Application Publication
(10) Pub. No.: US 2023/0348970 A1
(43) Pub. Date: Nov. 2, 2023

(54) FUNCTIONALIZED NANOSTRUCTURES AND FLOW CELL DEPRESSIONS

(71) Applicant: ILLUMINA CAMBRIDGE LIMITED, Cambridge (GB)

(72) Inventors: Gianluca Andrea Artioli, Cambridge (GB); Luca Redivo, Hitchin (GB); Alexandre Richez, Cambourne (GB); Xavier von Hatten, Cambridge (GB)

(21) Appl. No.: 18/176,761

(22) Filed: Mar. 1, 2023

Related U.S. Application Data
(60) Provisional application No. 63/315,353, filed on Mar. 1, 2022.

(51) Int. Cl. C12Q 1/6874; B01L 3/00; C08F 292/00

(52) U.S. Cl. CPC C12Q 1/6874; B01L 3/502761; C08F 292/00; C12Q 1/6806

(57) ABSTRACT

A functionalized nanostructure includes a metal nanostructure; an un-cleavable first primer and a cleavable second primer attached to a first region of the metal nanostructure through i) a first thiol linkage attached to a first polymer chain having a first polarity or ii) respective first thiol linkages attached to respective first polymer chains having the first polarity; and a cleavable first primer and an un-cleavable second primer attached to a second region of the metal nanostructure through i) a second thiol linkage attached to a second polymer chain having a second polarity different from the first polarity or ii) respective second thiol linkages attached to respective second polymer chains having the second polarity.

### CROSS-REFERENCE TO RELATED APPLICATION

This application claims the benefit of U.S. Provisional Application Ser. No. 63/315,353, filed Mar. 1, 2022, the contents of which is incorporated by reference herein in its entirety.

### REFERENCE TO SEQUENCE LISTING

The Sequence Listing submitted herewith is hereby incorporated by reference in its entirety. The name of the file is ILI231B_IP-2248-US_Sequence_Listing.xml, the size of the file is 19,892 bytes, and the date of creation of the file is Feb. 27, 2023.

### BACKGROUND

Various protocols in biological or chemical research involve performing a large number of controlled reactions on local support surfaces or within predefined reaction chambers. The designated reactions may then be observed or detected and subsequent analysis may help identify or reveal properties of chemicals involved in the reaction. In some examples, the controlled reactions alter charge, conductivity, or some other electrical property, and thus an electronic system may be used for detection. In other examples, the controlled reactions generate fluorescence, and thus an optical system may be used for detection.

### SUMMARY

Disclosed herein are nanostructures and flow cell depressions that are functionalized for simultaneous paired-end sequencing. Each nanostructure is a metal nanostructure that has different primer sets attached to different regions on the surface. Each flow cell depression includes a metal film that has the different primer sets attached to different regions on the surface. Different polymer strands are used to attach the respective primer sets to the different regions. While all of the polymer strands have a thiol linkage that is capable of forming a displaceable bond with the metal surface, some of the polymer strands have a first polarity and some other of the polymer strands have a second polarity that is different than the first polarity. The differences in polarity enable the different polymer strands to achieve separation on the metal surface, which, in turn, enables the different primer sets to be spatially separated. Spatial separation of the different primer sets enables a cluster of forward library template strands to be generated in one region of the nanostructure or film and a cluster of reverse library template strands to be generated in another region of the nanostructure or film. During a sequencing operation, the signals from the respective library template strands are readily distinguishable, i.e., the signals from one region do not deleteriously affect the signals from the other region.

### BRIEF DESCRIPTION OF THE FIGURES

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A schematically illustrates one example of a method for making a first of two different primer-containing polymer chains;

FIG. 1B schematically illustrates one example of a method for making a second of the two different primer-containing polymer chains;

FIG. 2 schematically illustrates the formation of one example of a functionalized nanostructure using the primer-containing polymer chains of FIG. 1A and FIG. 1B;

FIG. 3A schematically illustrates another example of a method for making a first of two different primer-containing polymer chains;

FIG. 3B schematically illustrates another example of a method for making a second of the two different primer-containing polymer chains;

FIG. 4 schematically illustrates the formation of another example of a functionalized nanostructure using the primer-containing polymer chains of FIG. 3A and FIG. 3B;

FIG. 5 schematically illustrates still another example of a method for making two different primer-containing polymer chains;

FIG. 6 schematically illustrates the formation of another example of a functionalized nanostructure using the primer-containing polymer chains of FIG. 5;

FIG. 7A schematically illustrates yet another example of a method for making a first of two different primer-containing polymer chains;

FIG. 7B schematically illustrates yet another example of a method for making a second of two different primer-containing polymer chains;

FIG. 8A and FIG. 8B together schematically illustrate the formation of another example of a functionalized nanostructure using the polymer chains of FIG. 7A and FIG. 7B, where FIG. 8A illustrates the formation of a polymer functionalized metal nanostructure, and FIG. 8B illustrates the formation of an example of the functionalized nanostructure from the polymer functionalized metal nanostructure;

FIG. 9 schematically illustrates an example of two functionalized primer sets that are used in examples of the functionalized nanostructures or some of the flow cells disclosed herein;

FIG. 10 is a top view of an example flow cell;

FIG. 11A is an enlarged, cross-sectional view, taken along the 11A-11A line of FIG. 10, depicting one example the flow cell architecture including the functionalized nanostructures anchored to a lane;

FIG. 11B is an enlarged, cross-sectional view, taken along the 11B-11B line of FIG. 10, depicting another example the flow cell architecture including the functionalized nanostructures anchored to posts;

FIG. 11C is an enlarged, cross-sectional view, taken along the 11C-11C line of FIG. 10, depicting yet another example the flow cell architecture including the functionalized nanostructures anchored to depressions;

FIG. 12 is an enlarged, cross-sectional view, taken along the 12-12 line of FIG. 10, depicting yet another example the flow cell architecture including a functionalized transparent metal film in the depressions; and

FIG. 13 is a graph depicting Dynamic Light Scattering (DLS) intensity (%, Y axis) versus the size (nm, X axis) for different polymer coated metal nanoparticles.

### DETAILED DESCRIPTION

Each of the nanostructures and metal films disclosed herein is functionalized with different primer sets at different regions on the nanostructure or film surface. Different polymer chains with different polarities are used to attach the respective primer sets to the different regions. As such, each of the functionalized nanostructures or metal films includes the surface chemistry for seeding and clustering library templates across the nanostructure or film surface. The primer sets are controlled so that the cleaving (linearization) chemistry is orthogonal at the different regions, which enables a cluster of forward strands to be generated in one region of the nanostructure or film and a cluster of reverse strands to be generated in another region of the nanostructure or film. This enables simultaneous paired-end sequencing, where the forward strands and reverse strands are sequenced at the same time.

The functionalized nanostructures may be used in an off-flow cell workflow. Because the primer sets are attached to the nanostructures, off-flow cell template strand preparation and amplification can take place. Then, the pre-clustered nanostructures may be incorporated into a flow cell for sequencing. The flow cell that is to be used with the functionalized nanostructures includes capture sites that can anchor the functionalized nanostructures at predetermined locations along the substrate(s) of the flow cell. Because the primer sets are part of the functionalized nanostructures, the flow cell substrate is not exposed to primer grafting processes or other surface preparation processes. As such, the use of the functionalized nanostructures simplifies the flow cell substrate preparation process.

Other examples of the flow cell disclosed herein include the metal film and spatially separated primers sets in the depression of a flow cell. This particular flow cell is integrated over a solid-state imager, such as a complementary metal-oxide semiconductor (CMOS) imager that does not require a large optical assembly to detect the fluorescent emissions from each of the regions.

### Definitions

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, on, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

It is to be understood that the ranges provided herein include the stated range and any value or sub-range within the stated range, as if such values or sub-ranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value.

As used herein, the term “attached” refers to the state of two things being joined, fastened, adhered, connected or bound to each other, either directly or indirectly. As examples, bonds that form may be covalent or non-covalent. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions.

A “capture site”, as used herein, refers to portion of a flow cell substrate having been modified, chemically, magnetically or electrostatically, that allows for anchoring of a functionalized nanostructure. In an example, the capture site may include a chemical capture agent, a magnetic capture agent, or an electrostatic capture agent.

A “chemical capture agent” is a material, molecule or moiety that is capable of anchoring to a functional agent of a functionalized nanostructure via a chemical mechanism. One example chemical capture agent includes a capture nucleic acid (e.g., a capture oligonucleotide) that is complementary to at least a portion of a target nucleic acid attached to a functionalized nanostructure. Still another example chemical capture agent includes a member of a binding pair that is capable of binding to a second member of a binding pair that is attached to the functionalized nanostructure. Example binding pairs include a NiNTA (nickel-nitrilotriacetic acid) ligand and a histidine tag, or streptavidin or avidin and biotin, etc. Yet another example of the chemical capture agent is a chemical reagent that is capable of forming a hydrogen bond, or a covalent bond with the functionalized nanostructure. Covalent bonds may be formed, for example, through thiol-disulfide exchange, click chemistry, Diels-Alder, Michael additions, amine-aldehyde coupling, amine-acid chloride reactions, amine-carboxylic acid reactions, nucleophilic substitution reactions, etc. Some chemical capture agents may be light-triggered, i.e., activated to chemically bind to the chemical capture agent when exposed to light.

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

As used herein, the term “depression” refers to a discrete concave feature in a substrate having a surface opening that is at least partially surrounded by interstitial region(s) of the substrate. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc.

The term “each,” when used in reference to a collection of items, is intended to identify an individual item in the collection, but does not necessarily refer to every item in the collection. Exceptions can occur if explicit disclosure or context clearly dictates otherwise.

As used herein, the term “electrostatic capture agent” refers to a charged material that is capable of electrostatically anchoring a charged or reversibly charged functionalized nanostructure. For pre-clustered nanostructures, the attached template nucleic acid strands are negatively charged. As such, positively charged pads, e.g., made of silanes, polymers with azide functional groups, poly-lysine, polyimines (e.g., polyethyleneimine, polypropylene imine, etc.), and other positively charged materials, may be used as the electrostatic capture agent. Another example of an electrostatic capture agent is an electrode that can attract, when a proper voltage is applied, a reversibly chargeable functional group that is incorporated into the functionalized nanostructure. As examples, amines or carboxylic acids can be reversibly switched between a neutral and a charged species in response to a pH change, and the charged species can be attracted to the electrode. The amines or carboxylic acids may be functional groups of a polyacrylamide, poly(acrylic acid) copolymer, etc. that is coated on the metal particles.

As used herein, the term “flow cell” is intended to mean a vessel having a flow channel where a reaction can be carried out, an inlet for delivering reagent(s) to the flow channel, and an outlet for removing reagent(s) from the flow channel. In some examples, the flow cell enables the detection of the reaction that occurs in the chamber. For example, the flow cell may include one or more transparent surfaces allowing for the optical detection of arrays, optically labeled molecules, or the like within the flow channel.

As used herein, a “flow channel” or “channel” may be an area defined between two bonded components, which can selectively receive a liquid sample. In some examples, the flow channel may be defined between a substrate and a lid, and thus may be in fluid communication with one or more depressions defined in the substrate or capture sites positioned on the substrate. The flow channel may also be defined between two substrate surfaces that are bonded together.

A “functional agent” is a material, molecule or moiety that is capable of anchoring to a chemical capture site of a flow cell via a chemical mechanism. One example functional agent includes a target nucleic acid that is complementary to a capture nucleic acid (e.g., a capture oligonucleotide) on the flow cell. Still another example functional agent includes a member of a binding pair that is capable of binding to a second member of a binding pair that is attached to the flow cell.

As used herein, the term “interstitial region” refers to an area, e.g., of a substrate that separates depressions or capture sites. For example, an interstitial region can separate one depression of an array from another depression of the array. The two depressions that are separated from each other can be discrete, i.e., lacking physical contact with each other. In many examples, the interstitial region is continuous, whereas the depressions are discrete, for example, as is the case for a plurality of depressions defined in an otherwise continuous surface. The separation provided by an interstitial region can be partial or full separation. Interstitial regions may have a surface material that differs from the surface material of the depressions or capture sites defined in or on the surface. For example, depressions can have a transparent metal film and two different primer sets therein, and the interstitial regions can be free of the transparent metal film and primer sets.

As used herein, the term “magnetic capture agent” refers to a magnetic material that is capable of magnetically anchoring a functionalized nanostructure. Example magnetic capture agents include ferromagnetic materials and ferrimagnetic materials.

As used herein, the term “mechanism” refers to a functional agent, a magnetic material or a reversibly chargeable functional group that is incorporated into the metal nanostructure in order to render the functionalized nanostructures capable of anchoring to a capture site in a flow cell.

As used herein, a “nucleotide” includes a nitrogen containing heterocyclic base, a sugar, and one or more phosphate groups. Nucleotides are monomeric units of a nucleic acid sequence. In ribonucleic acids (RNA), the sugar is a ribose, and in deoxyribonucleic acids (DNA), the sugar is a deoxyribose, i.e., a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

The term “orthogonal,” when used to describe two functional groups, two cleaving chemistries, or two polarities, means that the groups, chemistries, or polarities are different from each other. Orthogonal functional groups are capable of reacting with different functional groups, e.g., an azide may be reacted with may be reacted with an alkyne or DBCO (dibenzocyclooctyne) while an amino may be reacted with an activated carboxylate group or an N-hydroxysuccinimide (NHS) ester. Orthogonal cleaving chemistries are susceptible to different cleaving agents so that the first cleaving chemistry is unaffected when exposed to the cleaving agent for the second cleaving chemistry, and the second cleaving chemistry is unaffected when exposed to the cleaving agent for the first cleaving chemistry. The term “orthogonal polarities” refers to the polarities of two different compounds (e.g., polymer chains), where the respective polarities are different enough that segregation of the compounds can be obtained. The further apart the polarities are, the better the separation.

As used herein, the term “primer” is defined as a single stranded nucleic acid sequence (e.g., single strand DNA). Some primers are part of a primer set, which serve as a starting point for template amplification and cluster generation. Other primers, referred to herein as sequencing primers, serve as a starting point for DNA synthesis. The 5′ terminus of each primer in a primer set may be modified to allow a coupling reaction with a functional group of a polymer chain. The primer length can be any number of bases long and can include a variety of non-natural nucleotides. In an example, the sequencing primer is a short strand, ranging from 10 to 60 bases, or from 20 to 40 bases.

The term “primer set” refers to a pair of primers that together enable the amplification of a template nucleic acid strand (also referred to herein as a library template). Opposed ends of the template strand include adapters to hybridize to the respective primers in a set. The term “functionalized primer set” refers to the pair of primers after they are attached to polymer chain(s).

The term “substrate” refers to a structure upon which various components of the flow cell (e.g., capture sites, transparent metal films, primer(s), etc.) may be added. The substrate may be a wafer, a panel, a rectangular sheet, a die, or any other suitable configuration. The substrate is generally rigid and is insoluble in an aqueous liquid. The substrate may be inert to the chemistry that is present in the depressions or that is captured at a capture site. For example, a substrate can be inert to chemistry used to attach the primer(s), used in sequencing reactions, etc. The substrate may be a single layer structure, or a multi-layered structure (e.g., including a support and a patterned resin on the support). Examples of suitable substrates will be described further herein.

The term “transparent” refers to a material, e.g., in the form of a layer, that is capable of transmitting a particular wavelength or range of wavelengths. For example, the material may be transparent to wavelength(s) that are used in a sequencing operation. Transparency may be quantified using transmittance, i.e., the ratio of light energy falling on a body to that transmitted through the body. The transmittance of a transparent layer will depend upon the thickness of the layer, the wavelength of light, and the dosage of the light to which it is exposed. In the examples disclosed herein, the transmittance of the transparent metal layer may range from 0.1 (10%) to 1 (100%). The material of the transparent metal layer may be a pure material, a material with some impurities, or a mixture of materials, as long as the resulting layer is capable of the desired transmittance.

Functionalized Nanostructures and Methods of Making Functionalized Nanostructures

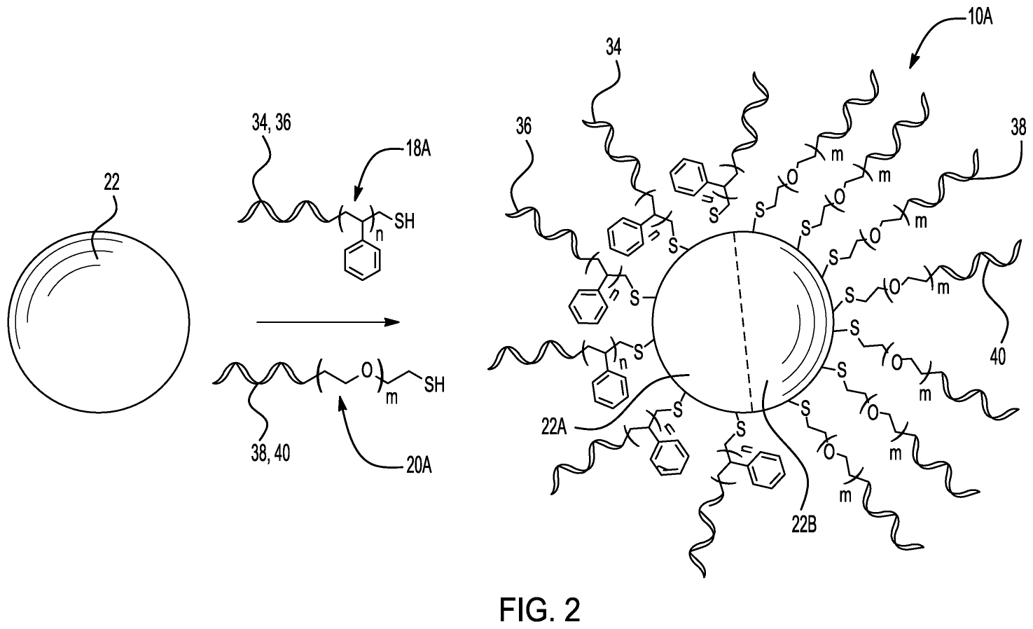

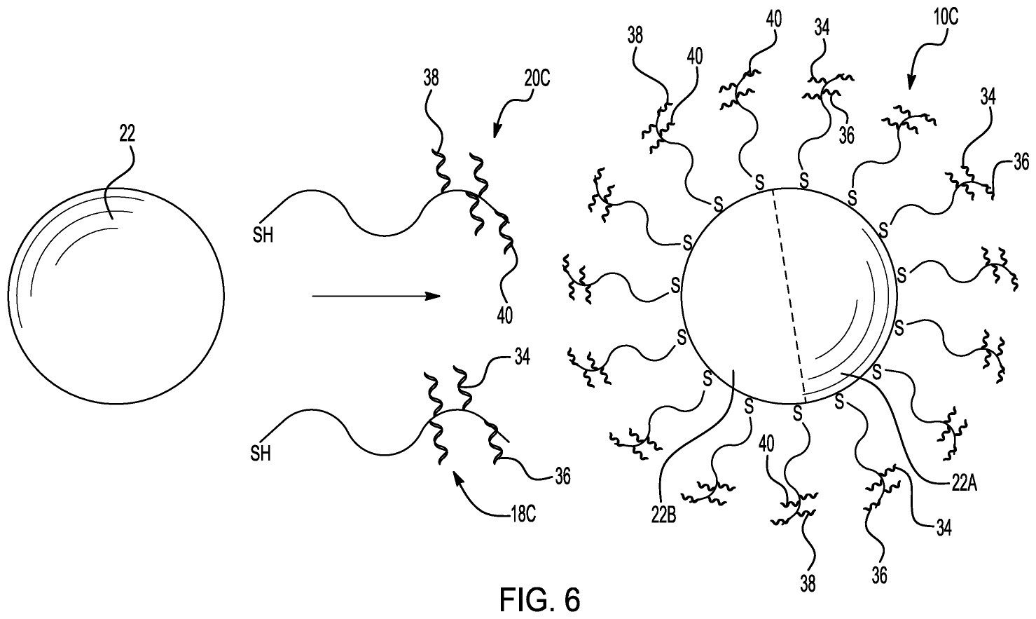

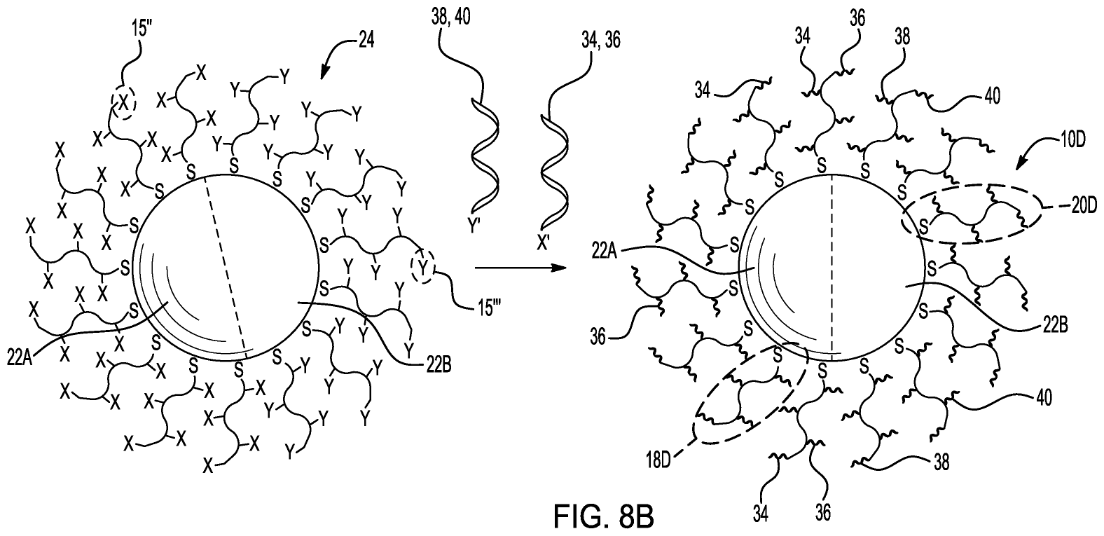

The functionalized nanostructures disclosed herein include a metal nanostructure; an un-cleavable first primer and a cleavable second primer attached to a first region of the metal nanostructure through i) a first thiol linkage attached to a first polymer chain having a first polarity or ii) respective first thiol linkages attached to respective first polymer chains having the first polarity; and a cleavable first primer and an un-cleavable second primer attached to a second region of the metal nanostructure through i) a second thiol linkage attached to a second polymer chain having a second polarity or ii) respective second thiol linkages attached to respective second polymer chains having the second polarity. Different examples of the functionalized nanostructures 10A, 10B, 10C, 10D are shown and described in reference to FIG. 2, FIG. 4, FIG. 6, and FIG. 8B.

Each of the functionalized nanostructures 10A, 10B, 10C, 10D includes a metal nanostructure 22 (shown at reference numeral 22 in FIG. 2, FIG. 4, FIG. 6, FIG. 8A, and FIG. 8B). The material of the metal nanostructure 22 may be any metal that is capable of forming a displaceable bond (i.e., can be broken and reformed) with the sulfur atom of a thiol group (—SH). In an example, the metal nanostructure 22 is selected from the group consisting of gold (Au), platinum (Pt), copper (Cu), silver (Ag), and alloys of any of these metals.

In an example, the metal nanostructure 22 is a spherical nanoparticle. In another example, the metal nanostructure 22 is a non-spherical nanoparticle, such as a cube, a triangular prism, rod shaped, a platelet, cage-like (e.g., non-spherical, hollow particles having a porous shell), a tube, etc. In still another example, the metal nanostructure 22 is an irregularly shaped nanoparticle.

The metal nanostructure 22 may have a solid structure, a hollow structure, or a core-shell structure.

When the core-shell structure is used, it is to be understood that one material is present at the interior, and the metal material is present at the exterior such that it at least partially encapsulates the interior. An example of the core material is a magnetic material (e.g., nickel, iron, cobalt, or other ferromagnetic materials, ferrites, magnetite, or other ferromagnetic materials, etc.). This example core-shell structure may be suitable for use when the flow cell substrate includes a magnetic capture agent, because the magnetic material is the mechanism for attachment to the flow cell capture site.

The dimensions of the metal nanostructure 22 may vary depending upon its shape. In the examples disclosed herein, the largest dimension (e.g., diameter, length, median, etc.) of the metal nanostructure 22 is on the nanoscale, and thus ranges from about 1 nm to less than 1000 nm. In some examples, the metal nanostructures 22 are nanoparticles having a diameter of greater than or equal to 1 nm, 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, or greater than or equal to 100 nm.

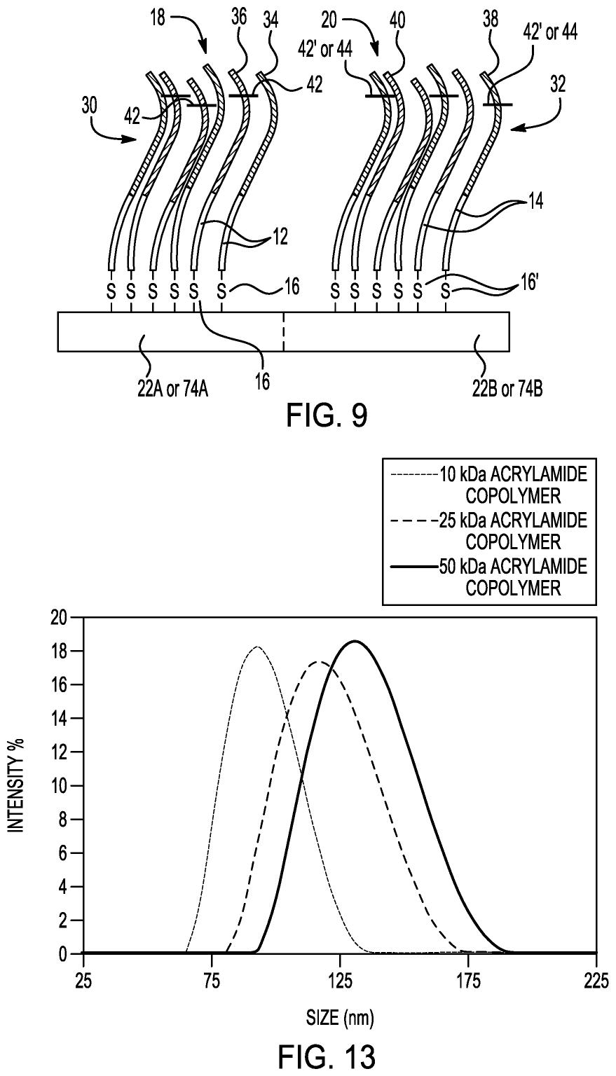

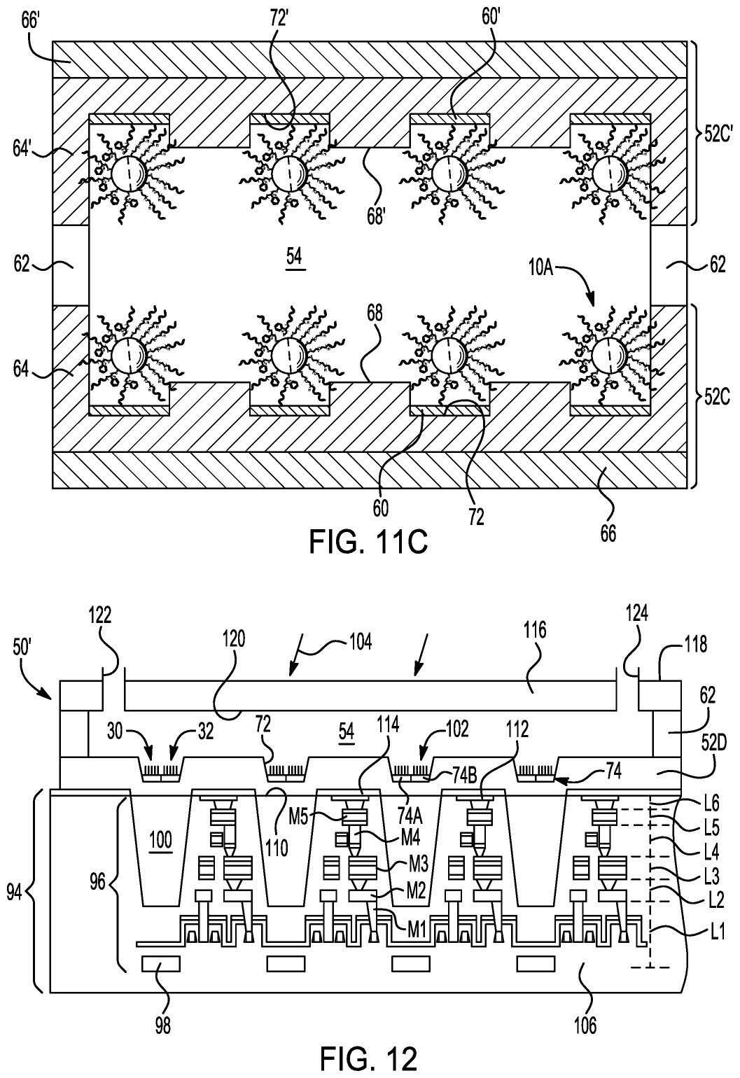

Each of the functionalized nanostructures 10A, 10B, 10C, 10D also includes different primer sets attached to different regions 22A, 22B (shown at reference numerals 22A, 22B in FIG. 2, FIG. 4, FIG. 6, FIG. 8A, and FIG. 8B) of the metal nanostructure 22. The un-cleavable first primer and cleavable second primer are part of one primer set, and the cleavable first primer and the un-cleavable second primer are part of another primer set. FIG. 9 depicts an example of the functionalized primer sets 30, 32 that are attached to the different regions 22A, 22B of the metal nanostructure 22, or as described in further detail below in reference to FIG. 12, to different regions 74A, 74B of a transparent metal film 74.

The first functionalized primer set 30 includes the un-cleavable first primer 34 and the cleavable second primer 36; and the second functionalized primer set 32 includes the cleavable first primer 38 and the un-cleavable second primer 40.

The un-cleavable first primer 34 and the cleavable second primer 36 of the first functionalized primer set 30 are oligonucleotide pairs, e.g., where the un-cleavable first primer 34 is a forward amplification primer and the cleavable second primer 36 is a reverse amplification primer or where the cleavable second primer 36 is the forward amplification primer and the un-cleavable first primer 34 is the reverse amplification primer. The cleavable second primer 36 includes a cleavage site 42, while the un-cleavable first primer 34 does not include a cleavage site 42.

The cleavable first primer 38 and the un-cleavable second primer 40 of the second functionalized primer set 32 are also oligonucleotide pairs, e.g., where the cleavable first primer 38 is a forward amplification primer and the un-cleavable second primer 40 is a reverse amplification primer or where the un-cleavable second primer 40 is the forward amplification primer and the cleavable first primer 38 is the reverse amplification primer. The cleavable first primer 38 includes a cleavage site 42′ or 44, while the un-cleavable second primer 40 does not include a cleavage site 42′ or 44.

It is to be understood that the un-cleavable first primer 34 of the first functionalized primer set 30 and the cleavable first primer 38 of the second functionalized primer set 32 have the same nucleotide sequence (e.g., both are forward amplification primers), except that the cleavable first primer 38 includes the cleavage site 42′ or 44 integrated into the nucleotide sequence or into a linker that attaches the nucleotide sequence to the polymer chain 14. Similarly, the cleavable second primer 36 of the first functionalized primer set 30 and the un-cleavable second primer 40 of the second functionalized primer set 32 have the same nucleotide sequence (e.g., both are reverse amplification primers), except that the cleavable second primer 36 includes the cleavage site 42 integrated into the nucleotide sequence or into a linker that attaches the nucleotide sequence to the polymer chain 12.

It is to be understood that when the first primers 34 and 38 are forward amplification primers, the second primers 36 and 40 are reverse primers, and vice versa.

The un-cleavable primers 34, 40 may be any primers with a universal sequence for capture and/or amplification purposes, such as P5 and P7 primers, or any combination of PA, PB, PC, and PD primers (e.g., PA and PB or PA and PD, etc.).

Examples of the P5 and P7 primers are used on the surface of commercial flow cells sold by IIlumina Inc. for sequencing, for example, on HISEQ™, HISEQX™, MISEQ™, MISEQDX™, MINISEQ™, NEXTSEQ™, NEXTSEQDX™, NOVASEQ™, ISEQ™, GENOME ANALYZER™, and other instrument platforms. The P5 primer is:

un-cleavable P5: 5′ → 3′
(SEQ. ID. NO. 1)
AATGATACGGCGACCACCGAGACTACAC

The P7 primer may be any of the following:

un-cleavable P7 #1: 5′ → 3′
(SEQ. ID. NO. 2)
CAAGCAGAAGACGGCATACGAAT
un-cleavable P7 #2: 5′ → 3′
(SEQ. ID. NO. 3)
CAAGCAGAAGACGGCATACAGAT

The other primers (PA-PD) mentioned above include:

un-cleavable PA 5′ → 3′
(SEQ. ID. NO. 4)
GCTGGCACGTCCGAACGCTTCGTTAATCCGTTGAG
un-cleavable cPA (PA′) 5′ → 3′
(SEQ. ID. NO. 5)
CTCAACGGATTAACGAAGCGTTCGGACGTGCCAGC
un-cleavable PB 5′ → 3′
(SEQ. ID. NO. 6)
CGTCGTCTGCCATGGCGCTTCGGTGGATATGAACT
un-cleavable cPB (PB′) 5′ → 3′
(SEQ. ID. NO. 7)
AGTTCATATCCACCGAAGCGCCATGGCAGACGACG
un-cleavable PC 5′ → 3′
(SEQ. ID. NO. 8)
ACGGCCGCTAATATCAACGCGTCGAATCCGCAACT
cPC (PC′) 5′ → 3′
(SEQ. ID. NO. 9)
AGTTGCGGATTCGACGCGTTGATATTAGCGGCCGT
un-cleavable PD 5′ → 3′
(SEQ. ID. NO. 10)
GCCGCGTTACGTTAGCCGGACTATTCGATGCAGC
un-cleavable cPD (PD′) 5′ → 3′
(SEQ. ID. NO. 11)
GCTGCATCGAATAGTCCGGCTAACGTAACGCGGC

As noted, all of these primers are un-cleavable primers 34, 40 because they do not include a cleavage site 42, 42′, 44. It is to be understood that any suitable universal sequence can be used as the un-cleavable primers 34, 40.

Examples of cleavable primers 36, 38 include the P5 and P7 primers or other universal sequence primers (e.g., the PA, PB, PC, PD primers) and respective cleavage sites 42, 42′ or 42, 44. The cleavage sites 42, 42′ or 42, 44 may be incorporated into the sequence of the respective primer 36, 38 or into the linker that attaches the cleavable primers 36, 38 to the respective polymer chains 12, 14. Examples of suitable cleavage sites 42, 42′, 44 include enzymatically cleavable nucleobases or chemically cleavable nucleobases, or modified nucleobases. Some specific examples of the cleavage sites 42, 42′, 44 include uracil, 8-oxoguanine, allyl-T, allyl ether, disulfide, a vicinal diol, and a restriction enzyme site. The cleavage sites 42, 42′, 44 may be incorporated at any point in the primer strand or in the linker.

Some specific examples of the cleavable primers 36, 38 are shown below, where the cleavage site 42, 42′, 44 is uracil or “n”:

cleavable P5: 5′ → 3′
(SEQ. ID. NO. 12)
AATGATACGGCGACCACCGAGAUCTACAC
cleavable P5: 5′ → 3′
(SEQ. ID. NO. 13)
AATGATACGGCGACCACCGAGAnCTACAC

wherein “n” is allyl T,
The cleavable P7 primer may be any of the following:

P7 #1: 5′ → 3′
(SEQ. ID. NO. 14)
CAAGCAGAAGACGGCATACGAUAT
P7 #2: 5′ → 3′
(SEQ. ID. NO. 15)
CAAGCAGAAGACGGCATACGAnAT
P7 #3: 5′ → 3′
(SEQ. ID. NO. 16)
CAAGCAGAAGACGGCATACnAGAT
P7 #4:5′ → 3′
(SEQ. ID. NO. 17)
CAAGCAGAAGACGGCATACnAnAT

where “n” is 8-oxoguanine in each of SEQ. ID. NOS. 15-17.

In one example, the same type of cleavage site 42, 42′ is used in the cleavable primers 36, 38 of the respective primer sets 30, 32. As an example, the cleavage sites 42, 42′ are uracil bases, and the cleavable primers 36, 38 are P5U and P7U. The uracil bases or other cleavage sites may also be incorporated into any of the PA, PB, PC, and PD primers to generate the cleavable primers 36, 38. In this particular example, the un-cleavable primer 34 of the oligonucleotide pair 34, 36 may be P7, and the un-cleavable primer 40 of the oligonucleotide pair 38, 40 may be P5. Thus, in this example, the first primer set 30 includes P7, P5U and the second primer set 32 includes P5, P7U. This example primer set 30, 32 has the same linearization chemistry (cleavage sites 42, 42′) on opposite primers 36, 38; which, after amplification, cluster generation, and linearization, allows forward template strands to be formed on one region 22A or 74A, and reverse strands to be formed on the other region 22B, 74B. In this particular example, linearization can be achieved in both regions 22A, 22B or 74A, 74B simultaneously because the cleavage sites 42, 42′ are the same. In an example, a USER enzyme may be used for linearization of P5U and P7U.

In another example, a different type of cleavage site 42, 44 is used in the cleavable primers 36, 38 of the respective primer sets 30, 32. As examples, two different enzymatic cleavage sites may be used, two different chemical cleavage sites may be used, or one enzymatic cleavage site and one chemical cleavage site may be used. Examples of different cleavage sites 42, 44 that may be used in the respective cleavable primers 36, 38 include any combination of the following: vicinal diol, uracil, allyl ether, disulfide, restriction enzyme site, and 8-oxoguanine. This example primer set 30, 32 has the different linearization chemistry (cleavage sites 42, 44) on opposite primers 36, 38; which, after amplification, cluster generation, and two different linearization processes, allows forward template strands to be formed on one region 22A or 74A, and reverse strands to be formed on the other region 22B, 74B. In this particular example, linearization is sequentially performed in the respective regions 22A, 22B or 74A, 74B because the cleavage sites 42, 44 are orthogonal.

In addition to the primer pairs 34, 36 and 38, 40, each of the functionalized primer sets 30, 32 includes polymer chains 12, 14 that are to attach to a respective region 22A, 22B of the metal nanostructure 22 or a respective region 74A, 74B of the transparent metal film 74. The surface of the metal nanostructure 22 or the transparent metal film 74 includes functional groups (e.g., Au, Pt, Ag) that can react with the thiol linkages 16, 16′ that are respectively attached to the primers 34, 36 and 38, 40 through the first polymer chains 12 and the second polymer chains 14.

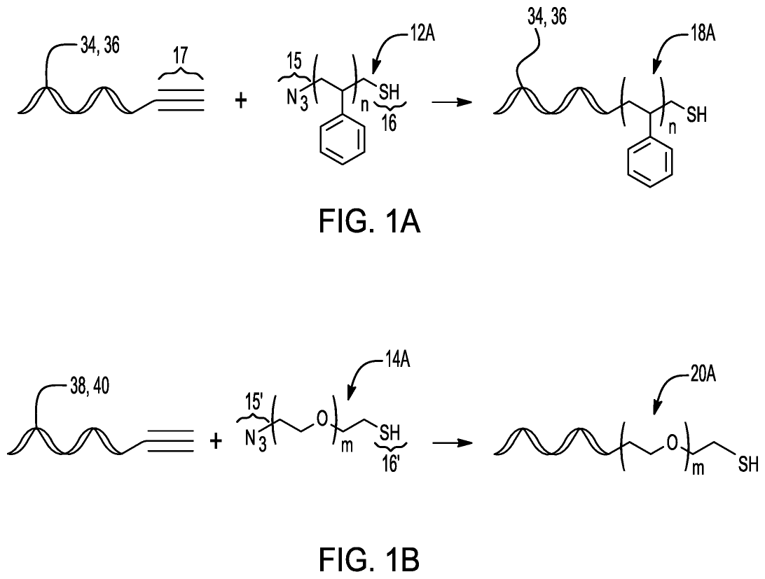

Different examples of the first and second polymer chains 12, 14 are described in reference to FIGS. 1A and 1B, FIGS. 3A and 3B, FIG. 5, and FIG. 7A and FIG. 7B. In each example, the polarity of the first polymer chains 12 is different from the polarity of the second polymer chains 14 so that the chains 12, 14 (and the respective primer sets 30, 32 attached thereto) separate during attachment to the metal nanostructure 22 or the transparent metal film 74. Examples of first and second polymer chains 12, 14 that have different polarities and that can be used together in the examples disclosed herein include: polystyrene and poly(ethylene glycol); and an acrylamide copolymer with N-isopropylacrylamide in at least some side chains and another acrylamide copolymer with ethylene oxide in at least some of the side chains. Each of the polystyrene and the poly(ethylene glycol) may have a weight average molecular weight ranging from about 1 kDa to about 100 kDa. Other suitable first and second polymer chains 12, 14 that have different polarities including any hydrophilic and hydrophobic polymer pair. Examples of suitable hydrophilic polymers include polyacrylamide, polyurethanes, poly-(hydroxyethyl methacrylamide), poly(ethylene glycol (ethylene glycol)-co-poly-ethyleneoxide)], polysaccharides, poly(peptides), polyimines, etc. Any of these hydrophilic polymers may be pair with a hydrophobic polymer, such as polyacrylics, polyethers, fluorocarbons, polyvinyls, poly(pyrrolidone), poly(dimethyl sulfoxide), etc.

Each of the first and second polymer chains 12, 14 includes the thiol linkage 16, 16′ at one of its terminal ends. The thiol linkages 16, 16′ provide the polymer chains 12, 14 with immobilization chemistry for attachment to the metal nanostructure 22 or the transparent metal film 74.

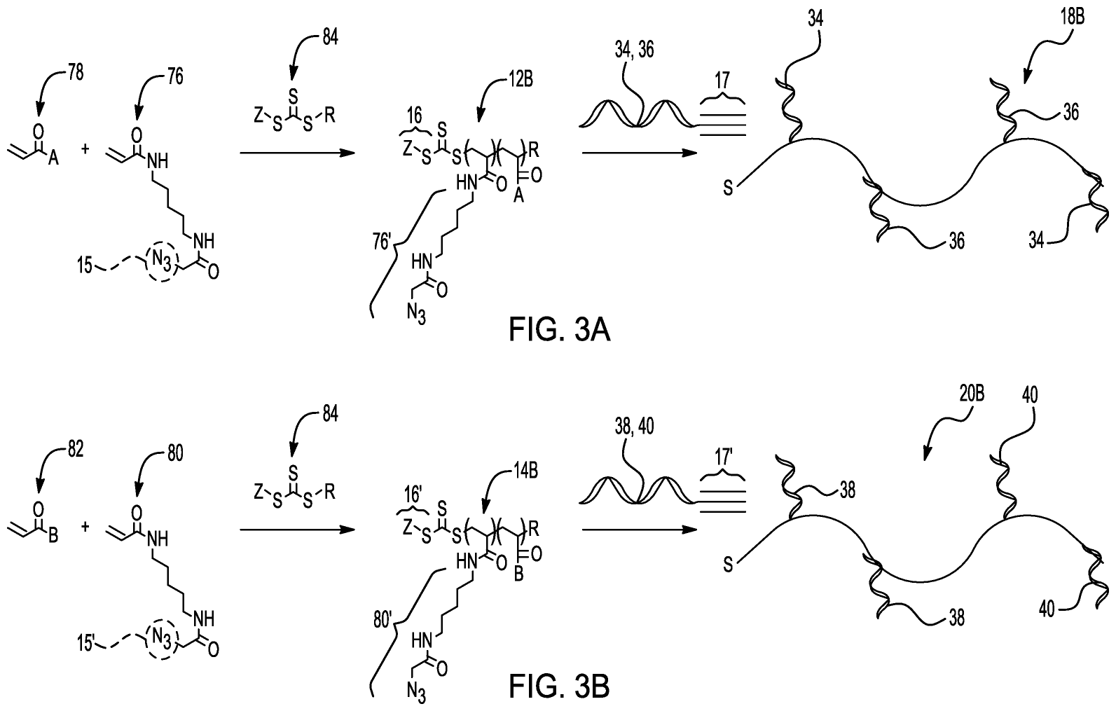

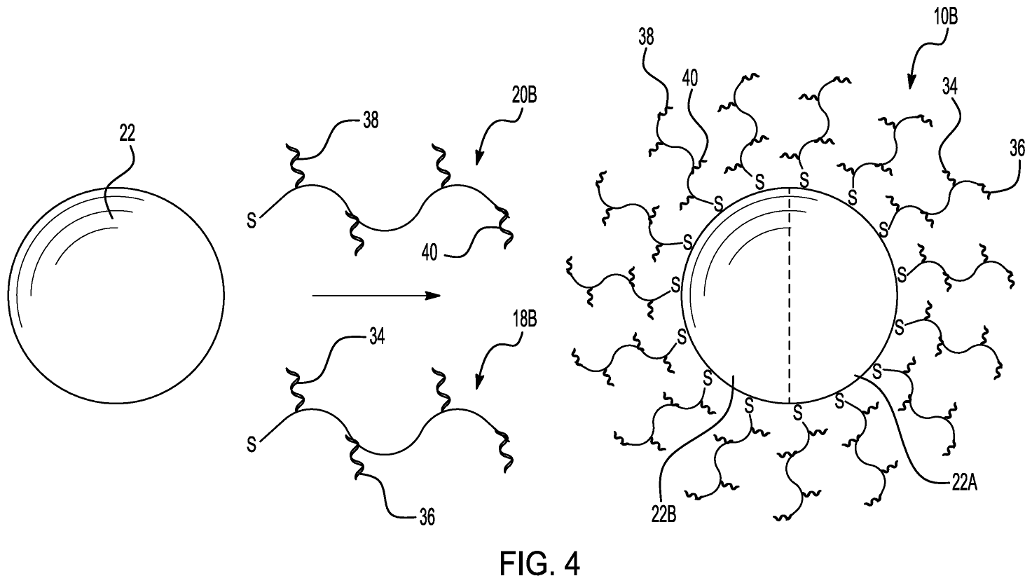

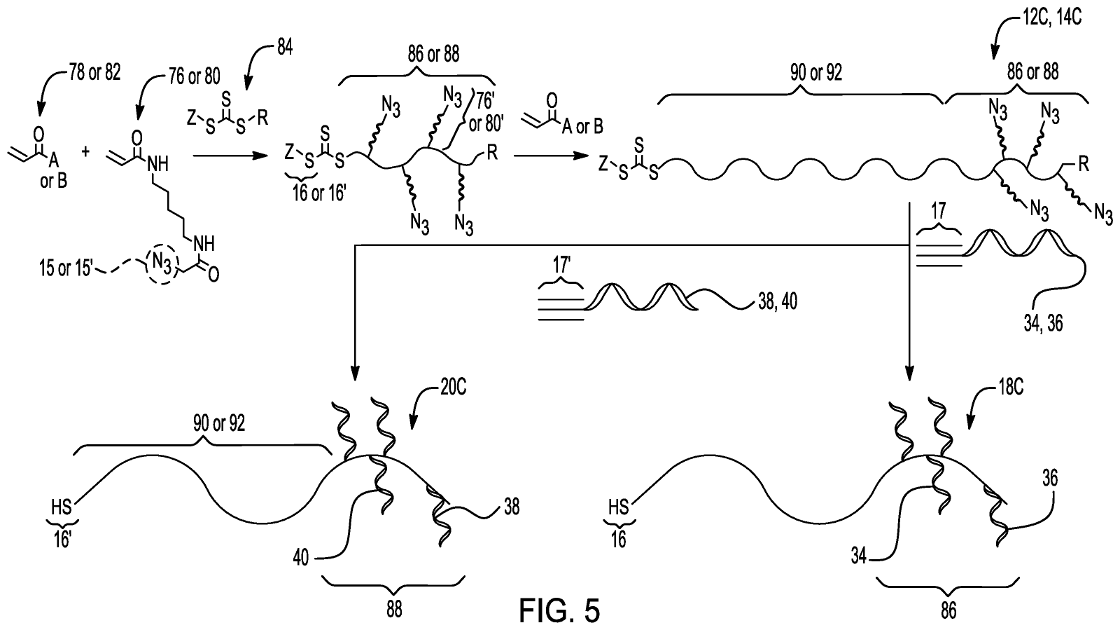

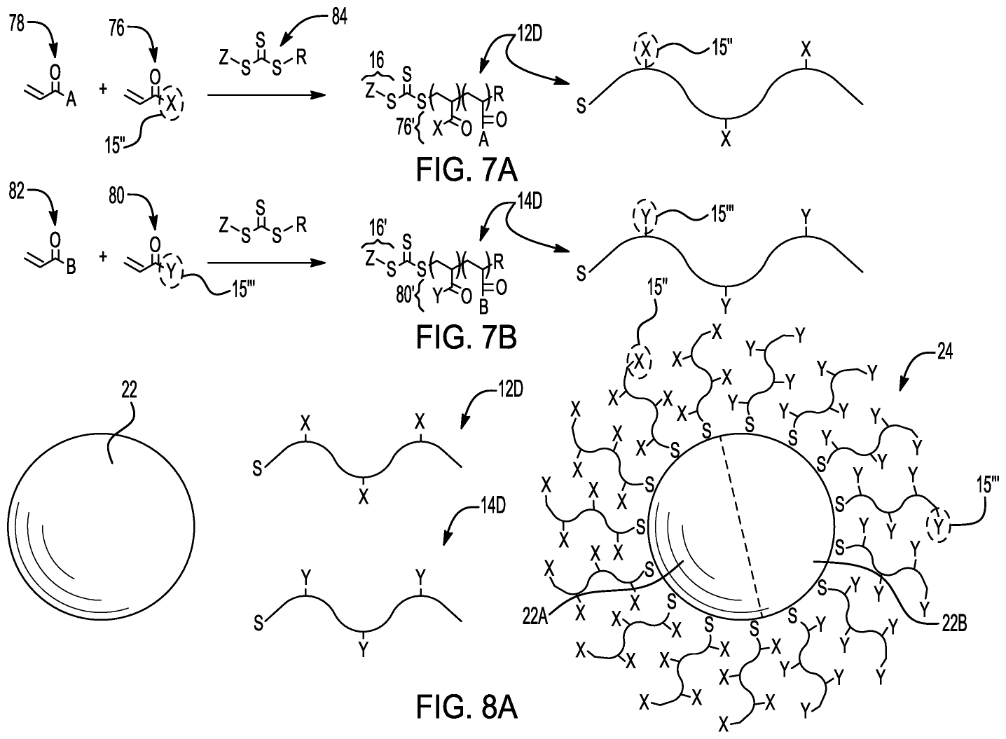

Each of the first polymer chains 12 also includes a functional group that can immobilize the linker attached to the 5′ end of the primers 34, 36. In some examples, as shown in FIG. 1A and FIG. 9, this functional group may be positioned at the other terminal end (opposed to the thiol linkage 16) of the polymer chain 12. In these examples, one primer 34, 36 is attached to one polymer chain 12 to form one example of the primer-containing polymer chain 18 (e.g., 18A in FIG. 1A), which can be attached to the metal nanostructure 22 or the transparent metal film 74 through the thiol linkage 16. In other examples, as shown in FIG. 3A, FIG. 5, and FIG. 7A, a plurality of the functional groups may be positioned in respective side chains of the polymer chain 12. In these examples, several primers 34, 36 are attached to one polymer chain 12 to form another example of the primer-containing polymer chain 18 (e.g., 18B in FIG. 3A, 18C in FIGS. 5, and 18D in FIG. 8B), which can be attached to the metal nanostructure 22 or the transparent metal film 74 through the thiol linkage 16.

Each of the second polymer chains 14 also includes a functional group that can immobilize the linker attached to the 5′ end of the primers 38, 40. In some examples, as shown in FIG. 1B and FIG. 9, this functional group may be positioned at the other terminal end (opposed to the thiol linkage 16′) of the polymer chain 14. In these examples, one primer 38, 40 is attached to one polymer chain 14 to form one example of the primer-containing polymer chain 20 (e.g., 20A in FIG. 1A), which can be attached to the metal nanostructure 22 or the transparent metal film 74 through the thiol linkage 16′. In other examples, as shown in FIG. 3B, FIG. 5, and FIG. 7B, a plurality of the functional groups may be positioned in respective side chains of the polymer chain 14. In these examples, several primers 38, 40 are attached to one polymer chain 14 to form another example of the primer-containing polymer chain 20 (e.g., 20B in FIG. 3B, 20C in FIGS. 5, and 20D in FIG. 8B), which can be attached to the metal nanostructure 22 or the transparent metal film 74 through the thiol linkage 16′.

In the examples shown in FIGS. 1A and 1B, FIGS. 3A and 3B, and FIG. 5, the linkers of the functionalized primer sets 30, 32 may be the same or different (orthogonal) and the functional group(s) of the polymer chains 12, 14 may be the same or different (orthogonal) because the primer-containing polymer chains 18, 20 are synthesized separately. In these examples, the terminal group of the linker that is attached to the 5′ end of each primer 34, 36 is selected to react with the functional group(s) of the polymer chains 12. Similarly, the terminal group of the linker that is attached to the 5′ end of each primer 38, 40 is selected to react with the functional group(s) of the polymer chains 14.

In the example shown in FIG. 8B, the linkers of the functionalized primer sets 30, 32 are different and the functional group(s) of the polymer chains 12, 14 are different because the primer-containing polymer chains 18, 20 are synthesized simultaneously. In these examples, the terminal group of the linker that is attached to the 5′ end of each primer 34, 36 is able to react with the functional group(s) of the polymer chains 12 but not with the functional group(s) of the polymer chains 14, and the terminal group of the linker that is attached to the 5′ end of each primers 38, 40 is selected to react with the functional group(s) of the polymer chain 14 but not with the functional group(s) of the polymer chains 12. In this example, the orthogonality of the chemistry linking the primers 34, 36 to the polymer chains 12 and the chemistry linking the primers 38, 40 to the polymer chains 14 ensures the separation of the primers 34, 36 and 38, 40 during attachment to the metal nanostructure 22 or the transparent metal film 74.

Examples of suitable linkers include nucleic acid linkers (e.g., 10 nucleotides or less) or non-nucleic acid linkers, such as a polyethylene glycol chain, an alkyl group or a carbon chain, an aliphatic linker with vicinal diols, a peptide linker, etc. An example of a nucleic acid linker is a polyT spacer, although other nucleotides can also be used. In one example, the spacer is a 6T to 10T spacer. One end of the linker is capable of attaching to the 5′ end of the primer 34, 36, 38, 40 and the other end of the linker is capable of reacting with the functional group(s) of the polymer chains 12 and/or 14. Examples of suitable linker end groups include an alkyne, an amine, a carboxylic acid, a tetrazine, an azide, or any other group that is orthogonal to the thiol linkage 16, 16′ (and thus will not react with the thiol linkages 16, 16′) and is capable of reacting with the functional group(s) (i.e., functional group 15, 15′ in FIG. 1A and FIG. 1B) of the polymer chains 12 and/or 14. While several examples have been provided, it is to be understood that other suitable linkers may be used.

The following are some examples of nucleotides including non-nucleic acid linkers with terminal alkyne groups (where B is the nucleobase and “oligo” is the primer sequence):

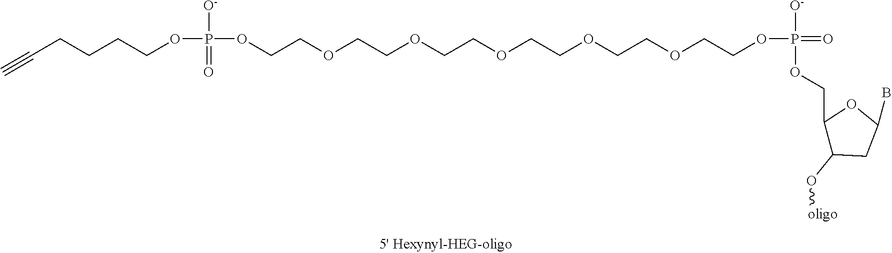

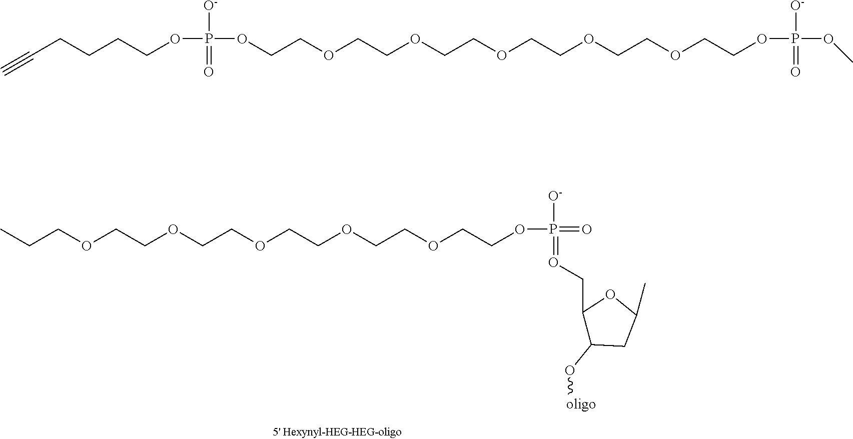

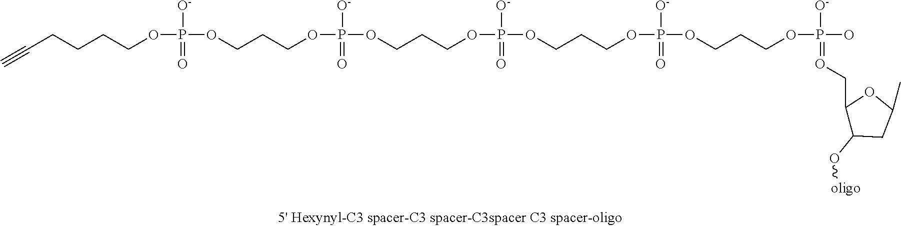

In each of these examples, the terminal alkyne is capable of reacting with an azide functional group of the polymer chain 12 and/or 14.

In any of the examples disclosed herein, the attachment of the functionalized primer sets 30, 32 to the respective regions 22A and 22B or 74A and 74B leaves a template-specific portion of the primers 34, 36 and 38, 40 free to anneal to its cognate template and the 3′ hydroxyl group free for primer extension.

Each of the functionalized nanostructures 10A, 10B, 10C, 10D may also include or be functionalized with a mechanism that is capable of anchoring to a capture site on a flow cell substrate. The mechanism may be chemical (e.g., a functional agent), electrostatic, or magnetic.

In some examples, the mechanism is a component of the functionalized nanostructures 10A, 10B, 10C, 10D that enables it to be anchored without further functionalization. For example, when the metal nanostructure 22 includes a magnetic material (as the mechanism) at its core, the functionalized nanostructures 10A, 10B, 10C, 10D may be anchored to a magnetic capture agent on the flow cell substrate. For another example, a reversibly chargeable functional group, such as an amine or a carboxylic acid, may be attached (e.g., through a thiol linkage) to the surface of the metal nanostructure 22 along with the functionalized primer sets 30, 32. In this example, the reversibly chargeable functional group (as the mechanism) enables the functionalized nanostructure 10A, 10B, 10C, 10D to be anchored to an electrostatic capture agent on the flow cell substrate. For still another example, the mechanism is a functional agent that is added to the functionalized nanostructure 10A, 10B, 10C, 10D that enables it to be anchored on the flow cell substrate. As one example, a target nucleic acid may be attached to the metal nanostructure 22 through a thiol linkage, where the target nucleic acid is complementary to a capture oligonucleotide on the flow cell substrate. As another example, a functional group for covalent attachment or a member of a binding pair may be attached to the metal nanostructure 22 through a thiol linkage.

FIG. 2, FIG. 4, FIG. 6, and FIGS. 8A and 8B each illustrate different example methods for generating the functionalized nanostructures 10A, 10B, 10C, 10D. Each of these methods will now be described.

The methods shown and described in reference to FIG. 2, FIG. 4, and FIG. 6 generally include: generating a first functionalized primer set 30 by attaching an un-cleavable first primer 34 and a cleavable second primer 36 to i) a first polymer chain 12 having a first polarity and a first thiol linkage 16 or ii) respective first polymer chains 12 having the first polarity and respective first thiol linkages 16; generating a second functionalized primer set 32 by attaching each of a cleavable first primer 38 and an un-cleavable second primer 40 to i) a second polymer chain 14 having a second polarity different from the first polarity and a second thiol linkage 16′ or ii) respective second polymer chains 14 having the second polarity and respective second thiol linkages; and attaching the first and second functionalized primer sets 30, 32, through the first thiol linkage 16 and the second thiol linkage 16′ or the respective first thiol linkages 16 and the respective second thiol linkages 16′, to spatially separate regions 22A, 22B of a metal surface. In the examples shown in FIG. 2, FIG. 4, and FIG. 6, the metal surface is the surface of the metal nanostructure 22. It is to be understood, however, that any of these methods may be used to attach the primer-containing polymer strands 18, 20 to the transparent metal film 74 in the depression 72 of a flow cell 50′.

FIG. 2 illustrates one example for making the functionalized nanostructure 10A. FIG. 1A and FIG. 1B illustrate the formation, respectively, of the primer-containing polymer chains 18A, 20A (which make up the functionalized primer sets 30, 32) used in the method of FIG. 2. In this example, generating the first functionalized primer set 30 involves reacting respective terminal end functional groups of the un-cleavable first primer 34 and of the cleavable second primer 36 with respective ends groups of the respective first polymer chains 12A (see FIG. 1A); and generating the second functionalized primer set 32 involves reacting respective terminal end functional groups of the cleavable first primer 38 and of the un-cleavable second primer 40 with respective ends groups of the respective second polymer chains 14A (FIG. 1B).

In FIG. 1A, the primers 34 and 36 are respectively reacted with the first polymer chains 12A. The first polymer chains 12A include the thiol linkage 16 at one terminal end of the backbone chain, and an orthogonal functional group 15 at the other terminal end of the backbone chain. The orthogonal functional group 15 is capable of reacting with the terminal end functional group 17 of the linker that is attached at the 5′ end of each primer 34, 36. In the example shown in FIG. 1A, the terminal alkyne of the primers 34, 36 is reacted, via click chemistry, with the terminal azide of the first polymer chains 12A. As other examples, a succinimidyl (NHS) ester terminated primer may be reacted with an amine orthogonal functional group 15, an aldehyde terminated primer may be reacted with a hydrazine orthogonal functional group 15, or an azide terminated primer may be reacted with an alkyne or DBCO (dibenzocyclooctyne) orthogonal functional group 15, or an amino terminated primer may be reacted with an activated carboxylate group or NHS ester orthogonal functional group 15. While several examples have been provided, it is to be understood that other chemistries may be used to attach the primers 34, 36 to the first polymer chains 12A.

While FIG. 1A depicts a single primer 34, 36 and a single polymer chain 12A being reacted, it is to be understood that both the un-cleavable first primer 34 and the cleavable second primer 36 are respectively reacted with polymer chains 12A to generate a plurality of the primer-containing polymer chains 18A, some of which include the un-cleavable first primers 34 and others of which include the cleavable second primers 36. Each of the primer-containing polymer chains 18A is part of the functionalized primer set 30.

In FIG. 1B, the primers 38 and 40 are respectively reacted with the second polymer chains 14A. The second polymer chains 14A include the thiol linkage 16′ at one terminal end of the backbone chain, and an orthogonal functional group 15′ at the other terminal end of the backbone chain. The orthogonal functional group 15′ is capable of reacting with the terminal end functional group 17 of the linker that is attached at the 5′ end of each primer 38, 40. In the example shown in FIG. 1B, the terminal alkyne of the primers 34, 36 is reacted, via click chemistry, with the terminal azide of the second polymer chains 14A. It is to be understood that other attachment chemistries may be used to attach the primers 38, 40 to the second polymer chains 14A.

While FIG. 1B depicts a single primer 38, 40 and a single polymer chain 14A being reacted, it is to be understood that both the cleavable first primer 38 and the un-cleavable second primer 40 are respectively reacted with polymer chains 14A to generate a plurality of the primer-containing polymer chains 20A, some of which include the cleavable first primers 38 and others of which include the un-cleavable second primers 40. Each of the primer-containing polymer chains 20A is part of the functionalized primer set 32.

The backbone chain of the first polymer chains 12A is selected to have the first polarity and the backbone chain of the second polymer chains 14A is selected to have a second polarity that is different from the first polarity. In the example shown in FIG. 1A and FIG. 1B, the first polymer chain 12A is polystyrene (where n ranges from 10 to 10,000), and the second polymer chain 14A is poly(ethylene glycol) (where m ranges from 10 to 10,000). In another example, the first polymer chain 12A is polystyrene (where n ranges from 100 to 1,000), and the second polymer chain 14A is poly(ethylene glycol) (where m ranges from 100 to 1,000).

In this example method, the first functionalized primer set 30 (i.e., primer-containing polymer chains 18A) and the second functionalized primer set 32 (i.e., primer-containing polymer chains 20A) are generated in separate reaction containers.

The method further includes mixing the first functionalized primer set 30 (i.e., primer-containing polymer chains 18A) and the second functionalized primer set 32 (i.e., primer-containing polymer chains 20A) to form a mixture; and exposing the mixture to the metal surface. This is schematically shown in FIG. 2. In this example, the metal surface includes the metal nanostructures 22, and exposing the mixture to the metal surface involves adding the metal nanostructures 22 to the mixture. In other examples (see FIG. 12), the metal surface includes a transparent metal film 74 positioned on a bottom surface in each of a plurality of depressions 72 of a flow cell 50′, and exposing the mixture to the metal surface involves incubating the mixture in each of the plurality of depressions 72. The functionalized primer sets 30, 32 may be mixed in an organic solvent or mixture of organic solvent, which may be selected based on the solubility of the polymer chain 12, 14 (with the thiol linkage 16, 16′) and the primers 34, 36, 38, 40. Any solvent or solvent mixture in which click chemistry is possible may be used. Some specific examples of suitable organic solvents include butanol, tetrahydrofuran (THF), dioxane, N-methyl-2-pyrrolidone (NMP), dimethylsulfoxide (DMSO), dimethylformamide (DMF), or the like. Alternatively, aqueous buffers may be used.

The mixture includes a predetermined ratio of the primer-containing polymer chains 18A (including primers 34 and 36) and the primer-containing polymer chains 20A (including primers 38 and 40). In some examples, the predetermined ratio is selected so that library template strands attached to the respective primers sets 30, 32 are able to generate a desirable fluorescence signal during sequencing. In these examples, the predetermined ratio ranges from 40:60 (2:3) to 60:40 (3:2), and in one example is 50:50 (1:1). For other applications, the predetermined ratio ranges from 10:90 (1:9) to 90:10 (9:1).

Within the mixture, the differing polarities contribute to the separation of the primer-containing polymer chains 18A from the primer-containing polymer chains 20A at the surfaces of the metal nanostructures 22. The thiol linkages 16, 16′ of the primer-containing polymer chains 18A and the primer-containing polymer chains 20A react (e.g., via adsorption) with the surface of the metal nanostructures 22.

In the final functionalized nanostructure 10A, it may be desirable for the total number of primer-containing polymer chains 18A and 20A per metal nanostructure 22 to range from about 100 to about 1,000. As such, the number of metal nanostructures 22 added to the mixture may depend upon the concentration of the primer-containing polymer chains 18A, 20A in the mixture. It may also be desirable for the total number of primer-containing polymer chains 18A and 20A per transparent metal film 74 to range from about 100 to about 1,000. As such, the concentration of the primer-containing polymer chains 18A, 20A in the mixture may depend upon the number of transparent metal films 74 in the flow cell 50′. In other examples, the total number of primer-containing polymer chains 18A and 20A per metal nanostructure 22 or metal film 74 ranges from about 200 to about 1,000.

As a result, the functionalized nanostructure 10A shown in FIG. 2 includes the un-cleavable first primer 34 and the cleavable second primer 36 attached to the first region 22A through the respective first thiol linkages 16 attached to the respective first polymer chains 12A, where the un-cleavable first primer 34 and the cleavable second primer 36 are attached to the respective first polymer chains 12A at respective ends (e.g., ends 15) that are opposed to the respective first thiol linkages 16; and the cleavable first primer 38 and the un-cleavable second primer 40 are attached to the second region 22B through the respective second thiol linkages 16′ attached to the respective second polymer chains 14A, where the cleavable first primer 38 and the un-cleavable second primer 40 are attached to the respective second polymer chains 14A at respective ends (e.g., ends 15′) that are opposed to the respective second thiol linkages 16′.

FIG. 4 illustrates an example for making the functionalized nanostructure 10B. FIG. 3A and FIG. 3B illustrate the formation, respectively, of the primer-containing polymer chains 18B, 20B (which make up the functionalized primer sets 30, 32) used in the method of FIG. 4. Generating the first functionalized primer set 30 involves polymerizing a first primer grafting monomer 76 and a first backbone monomer 78 to introduce primer reactive side chains 76′ to the first polymer chain 12B; and respectively grafting the un-cleavable first primer 34 and the cleavable second primer 36 to the primer reactive side chains 76′ of the first polymer chain 12B (FIG. 3A); and generating the second functionalized primer set 32 involves polymerizing a second primer grafting monomer 80 and a second backbone monomer 82 to introduce primer reactive side chains 80′ to the second polymer chain 14B; and respectively grafting the cleavable first primer 38 and the un-cleavable second primer 40 to the primer reactive side chains 80′ of the second polymer chain 14B.

Examples of suitable primer grafting monomers 76, 80 include any monomer that can be copolymerized with the respective backbone monomer 78, 82 and that includes the respective functional group 15, 15′, which is orthogonal to the thiol linkage 16, 16′ and is capable of reacting with the terminal end functional group 17, 17′ of the linker that is attached at the 5′ end of each primer 34, 36 or 38, 40.

One example of the primer grafting monomer 76, 80 is shown at structure (I):

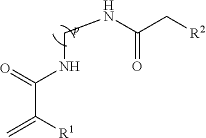

R¹ is selected from the group consisting of —H, a halogen, an alkyl, an alkoxy, an alkenyl, an alkynyl, a cycloalkyl, an aryl, a heteroaryl, a heterocycle, and optionally substituted variants thereof; R² (the primer grafting functional group 15, 15′) is selected from the group consisting of an azido, an amino, an alkenyl, an alkyne, a halogen, a hydrazone, a hydrazine, a carboxyl, a hydroxy, a tetrazole, a tetrazine, nitrile oxide, nitrone, and optionally substituted variants thereof; each (CH₂)ₚ can be optionally substituted; and p is an integer from 1 to 50. The primer grafting monomers 76, 80 shown in FIG. 3A and FIG. 3B are the monomer of structure (I), where R¹ is —H, R² is an azido, and p is 5.

It is to be understood that when R² of structure (I), is a halogen, the resulting polymer chain 12B, 14B may be exposed to NaN₃ and heating prior to primer grafting to replace the halogen with an azido.

Other examples of the primer grafting monomers 76, 80 that include tetrazine as the primer grafting functional group 15, 15′ are shown at structure (II):

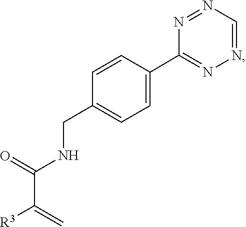

wherein R³ is H or a methyl, and structure (III):

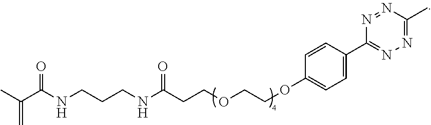

The tetrazine primer grafting functional group 15, 15′ is capable of binding to a bicyclo[6.1.0]nonyne (BCN)-terminated primer or a norbornene-terminated primer.

Still other examples of the primer grafting monomers 76, 80 include an activated ester as the primer grafting functional group 15, 15′. Examples include pentafluorophenyl acrylate, shown at structure (IV):

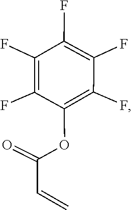 [SMILES: C=CC(=O)Oc1c(F)c(F)c(F)c(F)c1F]

pentafluorophenyl methacrylate, shown at structure (V):

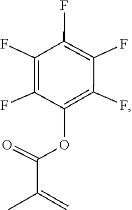 [SMILES: C=C(C)C(=O)Oc1c(F)c(F)c(F)c(F)c1F]

and vinyl dimethyl azlactone, shown at structure (VI):

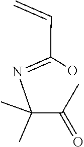

The activated ester primer grafting functional group 15, 15′ is capable of binding to an amine-terminated primer.

Still other examples of the primer grafting monomer 76, 80 including an azide as the primer grafting functional group 15, 15′ are shown at structure (VII):

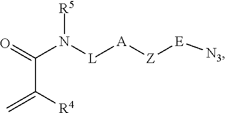

wherein R⁴ is hydrogen or an alkyl; R⁵ is hydrogen or an alkyl; L is a linker including a linear chain of 2 atoms to 20 atoms selected from the group consisting of carbon, oxygen, and nitrogen and optional substituents on the carbon and any nitrogen atoms in the chain; A is an N substituted amide having structure (VIII):

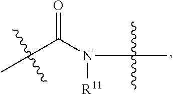

where R¹¹ is hydrogen or an alkyl; E is a linear chain of 1 atom to 4 atoms selected from the group consisting of carbon, oxygen and nitrogen, and optional substituents on the carbon and any nitrogen atoms in the chain; and Z is an optional nitrogen containing heterocycle. The azide primer grafting functional group 15, 15′ is capable of binding to an alkyne-terminated primer.

Examples of suitable backbone monomers 78, 82 include any monomer that can be copolymerized with the respective primer grafting monomer 76, 80 and includes a functional group (e.g., A and B shown in FIG. 3A and FIG. 3B, respectively) that is orthogonal to both the thiol linkage 16, 16′ and the terminal end functional group 17, 17′ of the linker that is attached at the 5′ end of each primer 34, 36 or 38, 40, and that imparts the desirable polarity to the polymer chains 12B, 14B.

Examples of the backbone monomers 78, 82 are shown at structure (IX):

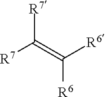

wherein three of R⁶, R^{6′}, R⁷ and R^{7′} are independently selected from the group consisting of —H, R⁸, —OR⁸, —C(O)OR⁸, —C(O)R⁸, —OC(O)R⁸, —C(O)NR⁹R¹⁰, and —NR⁹R¹⁰ and the fourth of R⁶, R^{6′}, R⁷ and R^{7′} is independently selected from the group consisting of styrene, N-isopropylacrylamide, and ethylene oxide; R⁸ is selected from the group consisting of —H, —OH, an alkyl, a cycloalkyl, a hydroxyalkyl, an aryl, a heteroaryl, a heterocycle, and optionally substituted variants thereof; and each of R⁹ and R¹⁰ is independently selected from the group consisting of —H, an alkyl, an alkylamino, an alkylamido, an aryl, a glycol, and optionally substituted variants thereof. It is to be understood that the fourth of R⁶, R^{6′}, R⁷ or R^{7′} in the monomer 78 imparts the first polarity and the fourth of R⁶, R^{6′}, R⁷ and R^{7′} in the monomer 82 imparts the second polarity that is different from the first polarity. Thus, these groups are selected to be different in the backbone monomers 78, 82.

As shown in FIG. 3A and FIG. 3B, the polymer chains 12B, 14B may each be synthesized using reversible addition-fragmentation chain transfer (RAFT) polymerization. RAFT polymerization allows the incorporation of a thiol chemical group (i.e., thiol linkages 16, 16′) on one end of the polymeric chain 12B, 14B. In addition, RAFT provides high control of the polymer dispersity and high compatibility with different monomers. These features allow the fabrication of a variety of polymer chains 12B, 14B with a single thiol end-group (i.e., thiol linkages 16, 16′).

RAFT polymerization of the monomer(s) 76 and 78 or 80 and 82 is initiated with a RAFT agent 84 (see both FIG. 3A and FIG. 3B). The RAFT agent 84 includes a thiocarbonylthio group (S═C—S) with substituents R and Z that impact the polymerization reaction kinetics and the degree of structural control. In the examples disclosed herein, the thiocarbonylthio group is a trithiocarbonate:

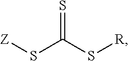

where the R group in the RAFT agent 84 is a free radical leaving group, and the Z group(s) control C═S bond reactivity and influence the rate of radical addition and fragmentation. Some examples of suitable RAFT agents for (meth)acrylate or (meth)acrylamide monomer polymerization include 2-(dodecylthiocarbonothioylthio)-2-methylpropionic acid, 4-cyano-4-[(dodecylsulfanylthiocarbonyl)sulfanyl]pentanoic acid, and 2-cyano-2-propyl dodecyl trithiocarbonate. 2-cyanomethyl dodecyl trithiocarbonate is a suitable RAFT agent for acrylate and acrylamide monomers. In one example, the RAFT agent is 4-cyano-4-[(dodecylsulfanylthiocarbonyl)sulfanyl]pentanoic acid. Any of these RAFT agents may be used to generate linear polymers.

For RAFT polymerization, the primer grafting monomers 76 or 80 and the backbone monomers 78 or 82 are mixed together at a predetermined mole ratio. In an example, the mole ratio of the primer grafting monomers 76 or 80 to the backbone monomers 78 or 82 may range from about 1:1 to about 1:100. As another example, the mole ratio of the primer grafting monomers 76 or 80 to the backbone monomers 78 or 82 may range from about 1:1 to about 1:49. The RAFT agent 84 may be added to this mixture at a concentration ranging from about 1 μM to about 10 M.

The mixture of the monomer(s) 76 and 78 or 80 and 82 and the RAFT agent 84 may be incorporated into a liquid, such as water and a co-solvent (e.g., N-methyl-2-pyrollidone (NMP), dimethyl formamide (DMF), dimethyl sulfoxide (DMSO), acetonitrile (MeCN), methanol (MeOH), ethanol (EtOH), isopropyl alcohol (IPA), dioxane, acetone, dimethylacetamide (DMAc), or the like). The liquid containing the mixture of monomers 76 and 78 or 80 and 82 and the RAFT agent 84 may also include a buffer to at least substantially prevent undesirable changes in the pH. The pH of the mixture may be acidic (<7). Examples of suitable buffers include TRIS (tris(hydroxymethyl)aminomethane or TRIZMA®), Bis-tris methane buffer, ADA buffer (a zwitterionic buffering agent), MES (2-ethanesulfonic acid), MOPS (3-(N-morpholino)propanesulfonic acid), or another acidic buffer.

The RAFT polymerization reaction may take place at a temperature ranging from about 50° C. to about 80° C. for a time ranging from about 1 hour to about 48 hours. At these reaction conditions, the weight average molecular weight of the resulting polymer chain may be up to 500 kDa. At lower temperatures (e.g., from about 25° C. to about 50° C.), the weight average molecular weight of the resulting polymer chain may be up to 10 kDa.

The process may incorporate the monomer(s) 76 and 78 or 80 and 82 randomly along the linear chain. Other monomer incorporation scenarios are possible, such as statistical, alternating, etc. With statistical incorporation, the sequential distribution of the monomeric units obeys known statistical laws. With alternating incorporation, the monomeric units are incorporated that they are alternating along the length. Because the primer grafting monomers 76, 80 are incorporated along the backbone of the polymer chain 12B, 14B, primer reactive side chains 76′, 80′ are respectively distributed along a backbone of the first polymer chain 12B and along a backbone of the second polymer chain 14B. The process also incorporates the thiol linkage 16, 16′ as one of the end groups in the respective polymer chains 12B, 14B.

Once the polymer chains 12B and 14B are generated, the primers 34, 36 are reacted with the functional groups 15 of the primer reactive side chains 76′ of the polymer chain 12B, and the primers 38, 40 are reacted with the functional groups 15′ of the primer reactive side chains 80′ of the polymer chain 14B. Because the primer reactive side chains 76′, 80′ are respectively distributed along the backbone of the first and second polymer chains 12B, 14B, the grafted primers 34, 36 and 38, 40 are also respectively distributed along the backbone of the first and second polymer chains 12B, 14B, as shown in FIG. 3A and FIG. 3B. While all of the functional groups 15, 15′ on a given polymer chain 12B, 14B may not be involved in a primer grafting reaction, a plurality of each primer 34 and 36 or 38 and 40 is grafted to each polymer chain 12B, 14B.

Because the amount of primers 34 and 36 or 38 and 40 present on each polymeric chain 12B, 14B can alter the polarity, each polymer chain 12B, 14B may include from about 0.25 mol % to about 100 mol % relative to a total number of moles in the polymeric chain 12B, 14B. In one example, there may be one primer 34 or 36, 38 or 40 per polymer chain 12B, 14B. A titration may be performed to confirm that the amount of primers 34 and 36 or 38 and 40 per chain is suitable to achieve the desired segregation and spatial separation of the two sets of primers 30, 32.

The molecular weight of the polymer chain 12B, 14B can also be selected to counteract any affect that primer polarity may have. For example, the hydrophobic polymer chain 12B or 14B may be at least as large (e.g., 10 kDa) as the primers 34, 36, 38, 40 (which are hydrophilic), and the hydrophilic polymer chain 14B or 12B may be smaller (e.g., 100 Da) than the primers 34, 36, 38, 40 because the primers 34, 36, 38, 40 will contribute to the hydrophilicity.

In this example method, the first functionalized primer set 30 (i.e., primer-containing polymer chains 18B) and the second functionalized primer set 32 (i.e., primer-containing polymer chains 20B) are generated in separate reaction containers.

The method further includes mixing the first functionalized primer set 30 (i.e., primer-containing polymer chains 18B) and the second functionalized primer set 32 (i.e., primer-containing polymer chains 20B) to form a mixture; and exposing the mixture to the metal surface. This is schematically shown in FIG. 4. In this example, the metal surface includes the metal nanostructures 22, and exposing the mixture to the metal surface involves adding the metal nanostructures 22 to the mixture. In other examples (see FIG. 12), the metal surface includes a transparent metal film 74 positioned on a bottom surface in each of a plurality of depressions 72 of a flow cell 50′, and exposing the mixture to the metal surface involves incubating the mixture in each of the plurality of depressions 72.

Any of the organic solvent(s) disclosed herein or an aqueous buffer may be used in the mixture. The mixture includes a predetermined ratio of the primer-containing polymer chains 18B (including primers 34 and 36) and the primer-containing polymer chains 20B (including primers 38 and 40). The predetermined ratio is selected so that library template strands attached to the respective primers sets 30, 32 are able to generate a desirable fluorescence signal during sequencing. In an example, the predetermined ratio ranges from 40:60 (2:3) to 60:40 (3:2), and in one example is 50:50 (1:1).

Within the mixture, the differing polarities contribute to the separation of the primer-containing polymer chains 18B from the primer-containing polymer chains 20B at the surfaces of the metal nanostructures 22. The thiol linkages 16, 16′ of the primer-containing polymer chains 18A and the primer-containing polymer chains 20A react (e.g., via adsorption) with the surface of the metal nanostructures 22.

In the final functionalized nanostructure 10B, it may be desirable for the total number of primer-containing polymer chains 18B and 20B per metal nanostructure 22 to range from about 100 to about 1,000. As such, the number of metal nanostructure 22 added to the mixture may depend upon the concentration of the primer-containing polymer chains 18B, 20B in the mixture. It may also be desirable for the total number of primer-containing polymer chains 18B and 20B per transparent metal film 74 to range from about 100 to about 1,000. As such, the concentration of the primer-containing polymer chains 18B, 20B in the mixture may depend upon the number of transparent metal films 74 in the flow cell 50′. In other examples, the total number of primer-containing polymer chains 18B and 20B per metal nanostructure 22 or metal film 74 ranges from about 200 to about 1,000.

As a result, the functionalized nanostructure 10B shown in FIG. 4 includes the un-cleavable first primer 34 and the cleavable second primer 36 attached to the first region 22A through the first thiol 16 linkage attached to the first polymer chain 12B, where the un-cleavable first primer 34 and the cleavable second primer 36 are attached to respective side chains 76′ of the first polymer chain 12B; and the cleavable first primer 38 and the un-cleavable second primer 40 attached to the second region 22B through the second thiol linkage 16′ attached to the second polymer chain 14B, where the cleavable first primer 38 and the un-cleavable second primer 40 are attached to the respective side chains 80′ of the second polymer chain 14B.

The method shown and described in reference to FIG. 3A, FIG. 3B, and FIG. 4 may be altered so that the side chains 76′ of the first polymer chain 12 are positioned closer to an end of the first polymer chain 12 that is opposed to the first thiol linkage 16 than to the first thiol linkage 16, and so that the side chains 80′ of the second polymer chain 14 are positioned closer to an end of the second polymer chain 14 that is opposed to the second thiol linkage 16′ than to the second thiol linkage 16′. This example method is shown and described in reference to FIG. 5 and FIG. 6. More particularly, FIG. 6 illustrates an example for making the functionalized nanostructure 10C. FIG. 5 illustrates the formation of the primer-containing polymer chains 18C, 20C (which make up the functionalized primer sets 30, 32) used in the method of FIG. 6.

In this example method, generating the first functionalized primer set 30 involves polymerizing a first primer grafting monomer 76 and a first backbone monomer 78 to form a first portion 86 of the first polymer chain 12C including primer reactive side chains 76′, continuing polymerization with the first backbone monomer 78 to form a second portion 90 of the first polymer chain 12C without primer reactive side chains 76′, and respectively grafting the un-cleavable first primer 34 and the cleavable second primer 36 to the primer reactive side chains 76′ of the first portion 86 of the first polymer chain 12C; and generating the second functionalized primer set 32 involves polymerizing a second primer grafting monomer 80 and a second backbone monomer 82 to form a first portion 88 of the second polymer chain 14C including primer reactive side chains 80′, continuing polymerization with the second backbone monomer 82 to form a second portion 92 of the second polymer chain 14C without primer reactive side chains 80′; and respectively grafting the cleavable first primer 38 and the un-cleavable second primer 40 to the primer reactive side chains 80′ of the first portion 88 of the second polymer chain 14C.

Any of the primer grafting monomers 76, 80 and any of the backbone monomers 78, 82 described in references to FIG. 3A, FIG. 3B, and FIG. 4 may be used in this example method. The functional groups A, B of the backbone monomers 78, 82 are selected to impart the desired different polarities of the resulting polymeric chains 12C, 14C.

To form the polymeric chains 12C, RAFT polymerization of the monomers 76 and 78 is initiated with the RAFT agent 84 described in reference to FIG. 3A and FIG. 3B. As described herein, the monomer mixture may include a liquid and buffer. This first step of the polymerization process is performed to introduce a desirable amount of primer reactive side chains 76′ into the portion 86 of the polymeric chain 12C. RAFT polymerization may take place at about 60° C. for a time ranging from about 2 hours to about 24 hours depending on the molecular weight and desired monomer conversion.

After the portion 86 is generated, RAFT polymerization may be continued with the monomer 78 (but without the monomer 76). This second step of the polymerization process introduces additional units of the backbone monomer 78 between the portion 86 including the primer reactive side chains 76′ and the thiol linkage 16. The additional units of the backbone monomer 78 create the portion 90 of the polymer chain 12C that does not include the primer reactive side chains 76′. RAFT polymerization may take place at about 60° C. for a time ranging from about 2 hours to about 24 hours depending on the molecular weight and desired monomer conversion.

To form the polymeric chains 14C, RAFT polymerization of the monomer(s) 80 and 82 is initiated with the RAFT agent 84 described in reference to FIG. 3A and FIG. 3B. As described herein, the monomer mixture may include a liquid and buffer. This first step of the polymerization process is performed to introduce a desirable amount of primer reactive side chains 80′ into the portion 88 of the polymeric chain 14C. RAFT polymerization may take place at about 60° C. for a time ranging from about 2 hours to about 24 hours depending on the molecular weight and desired monomer conversion.

After the portion 88 is generated, RAFT polymerization may be continued with the monomer 82 (but without the monomer 80). This second step of the polymerization process introduces additional units of the backbone monomer 82 between the portion 88 including the primer reactive side chains 80′ and the thiol linkage 16′. The additional units of the backbone monomer 82 create the portion 92 of the polymer chain 14C that does not include the primer reactive side chains 80′. As such, a predetermined portion (i.e., portion 92) of the backbone of the second polymer chain 14C that is adjacent to the second thiol linkage 16′ is free of the respective side chains 70′ of the second polymer chain 14C. The additional units of the backbone monomer 82 also contribute to the desired polarity of the polymeric chain 14C (which is different from the polarity of the polymer chain 12C). RAFT polymerization may take place at about 60° C. for a time ranging from about 2 hours to about 24 hours depending on the molecular weight and desired monomer conversion.

Once the polymer chains 12C and 14C are generated, the primers 34, 36 are reacted with the functional groups 15 of the primer reactive side chains 76′ of the polymer chain 12C, and the primers 38, 40 are reacted with the functional groups 15′ of the primer reactive side chains 80′ of the polymer chain 14C. Because the primer reactive side chains 76′, 80′ are positioned at an end of the backbone chain that is opposed to the end with the thiol linkages 16, 16′, the grafted primers 34, 36 and 38, 40 are also positioned at an end of the backbone chain that is opposed to the end with the thiol linkages 16, 16′, as shown in FIG. 5. While all of the functional groups 15, 15′ on a given polymer chain 12C, 14C may not be involved in a primer grafting reaction, a plurality of each primer 34 and 36 or 38 and 40 is grafted to each polymer chain 12C, 14C.

In this example method, the first functionalized primer set 30 (i.e., primer-containing polymer chains 18C) and the second functionalized primer set 32 (i.e., primer-containing polymer chains 20C) are generated in separate reaction containers.

The method further includes mixing the first functionalized primer set 30 (i.e., primer-containing polymer chains 18C) and the second functionalized primer set 32 (i.e., primer-containing polymer chains 20C) to form a mixture; and exposing the mixture to the metal surface. This is schematically shown in FIG. 6. In this example, the metal surface includes the metal nanostructures 22, and exposing the mixture to the metal surface involves adding the metal nanostructures 22 to the mixture. In other examples (see FIG. 12), the metal surface includes a transparent metal film 74 positioned on a bottom surface in each of a plurality of depressions 72 of a flow cell 50′, and exposing the mixture to the metal surface involves incubating the mixture in each of the plurality of depressions 72.

Any of the organic solvent(s) disclosed herein or an aqueous buffer may be used in the mixture. The mixture includes a predetermined ratio of the primer-containing polymer chains 18C (including primers 34 and 36) and the primer-containing polymer chains 20C (including primers 38 and 40). The predetermined ratio is selected so that library template strands attached to the respective primers sets 30, 32 are able to generate a desirable fluorescence signal during sequencing. In an example, the predetermined ratio ranges from 40:60 (2:3) to 60:40 (3:2), and in one example is 50:50 (1:1).

Within the mixture, the differing polarities contribute to the separation of the primer-containing polymer chains 18C from the primer-containing polymer chains 20C at the surfaces of the metal nanostructures 22. The distances between the thiol linkages 16, 16′ and the primers 34, 36 and 38, 40 in this example may aid in improving the segregation of the functionalized primer sets 30, 32. The thiol linkages 16, 16′ of the primer-containing polymer chains 18C and the primer-containing polymer chains 20C react (e.g., via adsorption) with the surface of the metal nanostructures 22.

In the final functionalized nanostructure 10C, it may be desirable for the total number of primer-containing polymer chains 18C and 20C per metal nanostructure 22 to range from about 100 to about 1,000. As such, the number of metal nanostructure 22 added to the mixture may depend upon the concentration of the primer-containing polymer chains 18C, 20C in the mixture. It may also be desirable for the total number of primer-containing polymer chains 18C and 20C per transparent metal film 74 to range from about 100 to about 1,000. As such, the concentration of the primer-containing polymer chains 18C, 20C in the mixture may depend upon the number of transparent metal films 74 in the flow cell 50′. In other examples, the total number of primer-containing polymer chains 18C and 20C per metal nanostructure 22 or metal film 74 ranges from about 200 to about 1,000.

As a result, the functionalized nanostructure 10C shown in FIG. 6 includes the un-cleavable first primer 34 and the cleavable second primer 36 attached to the first region 22A through the first thiol linkage 16 attached to the first polymer chain 12C, where the un-cleavable first primer 34 and the cleavable second primer 26 are attached to respective side chains 76′ of the first polymer chain 12C; the cleavable first primer 38 and the un-cleavable second primer 40 attached to the second region 22B through the second thiol linkage 16′ attached to the second polymer chain 14C, where the cleavable first primer 38 and the un-cleavable second primer 40 are attached to the respective side chains 80′ of the second polymer chain 14C; wherein each of the respective side chains 76′ of the first polymer chain 12C is positioned closer to an end of the first polymer chain 12C that is opposed to the first thiol linkage 16′ than to the first thiol linkage 16 and each of the respective side chains 80′ of the second polymer chain 14C is positioned closer to an end of the second polymer chain 14C that is opposed to the second thiol linkage 16′ than to the second thiol linkage 16′.

With monomer concentration and polymerization time, the example method shown in FIG. 5 and FIG. 6 enables control over the amount of primers 34 and 36 or 38 and 40 in each polymer chain 12C, 14C, the overall length of the polymer chains 12C, 14C, and the distance between the primers 34 and 36 or 38 and 40 and the thiol linkages 16, 16′.

FIG. 7A, FIG. 7B, FIG. 8A, and FIG. 8B illustrate still another example method for generating another example of the functionalized nanostructure 10D (see FIG. 8B). In this example method, the functional groups, shown at 15″, 15′″ of the polymer chains 12D, 14D are selected to be orthogonal to each other in addition to being orthogonal to the thiol linkages 16, 16′. This orthogonality ensures that the primers 34, 36 can attach to the functional groups 15″ but not the functional groups 15′″, and so that the primers 38, 40 can attach to the functional groups 15′″ but not the functional groups 15″. In this example then, the polymer chains 12D, 14D can be attached to the metal surface 22 (or 74) before the primers 34, 36 and 38, 40 grafted. Moreover, the primers 34, 36, 38, 40 can be grafted simultaneously to the polymer chains 12D, 14D after they are attached to the metal surface.

This example method generally includes respectively attaching first and second polymer chains 12D, 14D to spatially separate regions 22A, 22B (or 74A, 74B) of a metal surface, e.g., of metal nanostructure 22 or metal film 74, each first polymer chain 12D being attached to the metal surface through a first thiol linkage 16 and each first polymer chain 12D having a first polarity and a first functional group 15″ along a backbone of the first polymer chain 12D, and each second polymer chain 14D being attached to the metal surface through a second thiol linkage 16′, and each second polymer chain 14D having a second polarity and a second functional group 15′″ along a backbone of the second polymer chain 14D, the second functional group 15′″ being orthogonal to the first functional group 15″; respectively grafting an un-cleavable first primer 34 and a cleavable second primer 36 to the first polymer chains 12D through the first functional groups 15″; and respectively grafting a cleavable first primer 38 and an un-cleavable second primer 40 to the second polymer chains 14D through the second functional groups 15′″.

FIG. 7A and FIG. 7B schematically depict the formation of the polymer chains 12D and 14D. Separate RAFT polymerizations of the monomer(s) 76 and 78 or 80 and 82 are initiated with the RAFT agent 84 as described in reference to FIG. 3A and FIG. 3B, except that in this example the primer grafting functional groups 15″, 15′″ are specifically selected to be orthogonal to one another. Examples of orthogonal functional groups 15″, 15′″ include an activated ester functional group and an azide functional group, a tetrazine functional group and an activated ester functional group, an amine functional group and an azide functional group, a carboxyl functional group and an azide functional group, and a tetrazine functional group and an azide functional group. The backbone monomers 78, 82 are selected so that the respective functional groups A and B impart the desired polarity to the polymer chains 12D, 14D.

The process may incorporate the monomer(s) 76 and 78 or 80 and 82 randomly along the linear chain. Other monomer incorporation scenarios are possible, such as statistical, alternating, etc. With statistical incorporation, the sequential distribution of the monomeric units obeys known statistical laws. With alternating incorporation, the monomeric units are incorporated that they are alternating along the length. Because the primer grafting monomers 76, 80 are incorporated along the backbone of the polymer chain 12B, 14B, the primer reactive functional groups 15″, 15′″ (which may be part of a side chain depending upon the monomer 76, 80 that is used) are respectively distributed along a backbone of the first polymer chain 12D and along a backbone of the second polymer chain 14D. The process also incorporates the thiol linkage 16, 16′ as one of the end groups in the respective polymer chains 12D, 14D.

In this example method, the polymer chains 12D and 14D are generated in separate reaction containers.

The method further includes functionalizing the metal surface (e.g., metal nanostructure 22 or metal film 74) with the polymer chains 12D and 14D. The polymer chains 12D and 14D may be mixed together and exposed to the metal surface. This is schematically shown in FIG. 8A. In this example, the metal surface includes the metal nanostructures 22, and exposing the mixture to the metal surface involves adding the metal nanostructures 22 to the mixture. In other examples (see FIG. 12), the metal surface includes a transparent metal film 74 positioned on a bottom surface in each of a plurality of depressions 72 of a flow cell 50′, and exposing the mixture to the metal surface involves incubating the mixture in each of the plurality of depressions 72.

Any of the organic solvent(s) disclosed herein or an aqueous buffer may be used in the mixture. The mixture includes a predetermined ratio of the polymer chains 12D and 14D. In an example, the predetermined ratio ranges from 40:60 (2:3) to 60:40 (3:2), and in one example is 50:50 (1:1). Within the mixture, the differing polarities contribute to the separation of the polymer chains 12D and 14D at the surfaces of the metal nanostructures 22 (or metal film 74). The thiol linkages 16, 16′ of the polymer chains 12D and 14D react (e.g., via adsorption) with the surface of the metal surface. This generates the polymer functionalized metal nanostructure 24. Alternatively, this process can generate a polymer functionalized metal film.

This example method then involves simultaneously grafting the primers 34, 36 and 38, 40 to the respective polymer chains 12D, 14D. This is shown in FIG. 8B. To graft the primers 34, 36 and 38, 40, they may be mixed with the functionalized metal nanostructure 24. In this example, the primer 34, 36 and 38, 40 concentration will depend upon the percentage of the functional groups 15″, 15′″ on the polymer chains 12D, 14D. If the functional groups 15″, 15′″ are end groups, the ratio of primers 34 and 36 to functional groups 15″ may be 1:1 and the ratio of primers 38 and 40 to functional groups 15′″ may be 1:1. If the functional groups 15″, 15′″ are attached along the polymer chains 12D, 14D, the primers 34 and 36 and the primers 38 and 40 may occupy from 0.01 mol % to about 50 mol % of the respective polymer chains 12D, 14D. This mixture may also include water, a buffer, and a catalyst. The primers 34 and 36 attach to the polymer chains 12D and have no affinity for the orthogonal polymer chains 14D; and the primers 38 and 40 attach to the polymer chains 14D and have no affinity for the orthogonal polymer chains 12D. The selective affinity is due to the orthogonal functional groups 15″, 15′″.

Primer grafting generates the primer-containing polymer chains 18D, 20D and the functionalized metal nanostructure 10D.

Flow Cells for Use with the Functionalized Nanostructures

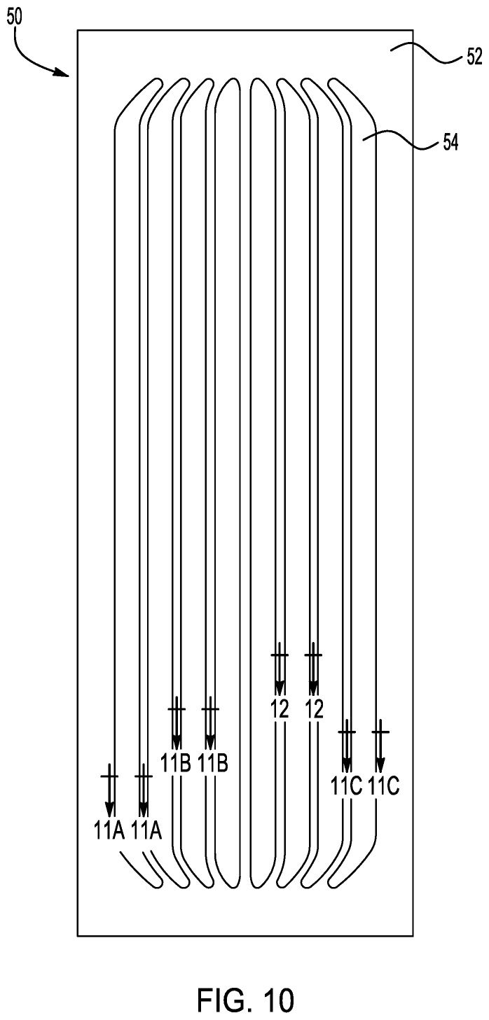

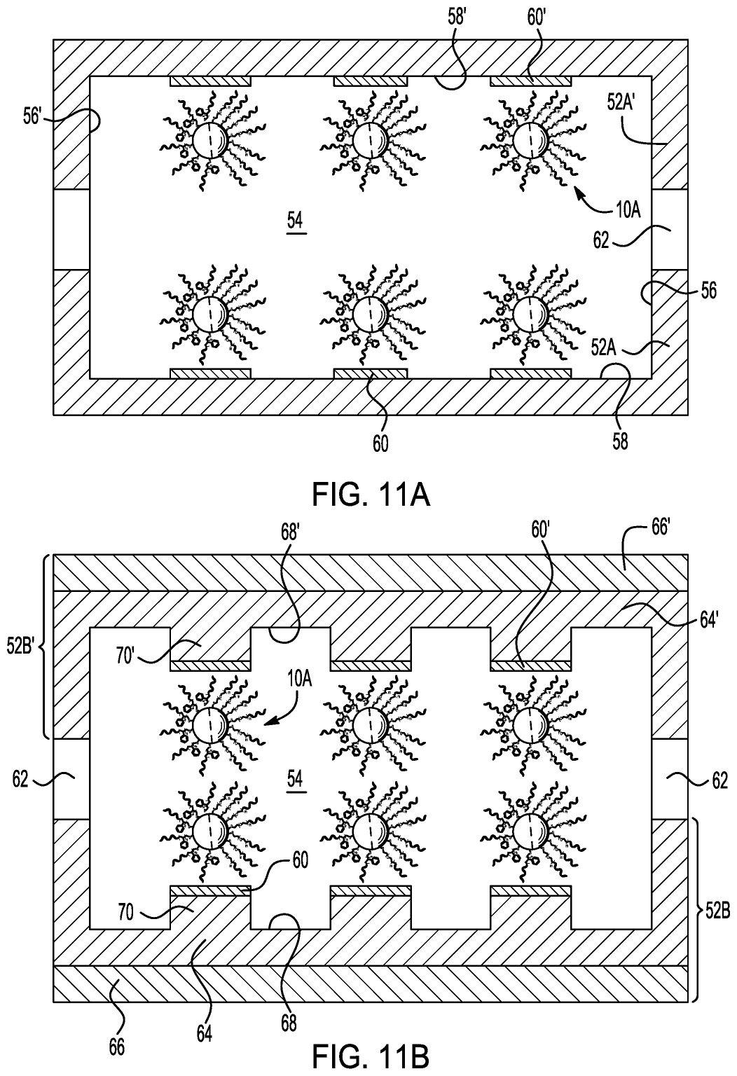

The functionalized nanostructures 10A, 10B, 10C, 10D may be used with any flow cell 50 (FIG. 10) that includes capture sites 60, 60′ (FIG. 11A, FIG. 11B, FIG. 11C). An example of the flow cell 50 is depicted from the top view in FIG. 10, and different examples of the flow cell architecture, including different configurations of the capture sites 60, 60′, are shown in FIG. 11A, FIG. 11B, and FIG. 11C.

A top view of an example of the flow cell 50 is shown in FIG. 10. As will be discussed in reference to FIG. 11A, FIG. 11B, and FIG. 11C, some examples of the flow cell 50 include two opposed substrates 52A, 52A′ or 52B, 52B′ or 52C, 52C′, each of which is configured with capture sites 60 or 60′. In these examples, a flow channel 54 is defined between the two opposed substrates 52A, 52A′ or 52B, 52B′ or 52C, 52C′. In other examples, the flow cell 50 includes one substrate 52A or 52B or 52C configured with capture sites 60 and a lid attached to the substrate 52A or 52B or 52C. In these examples, the flow channel 54 is defined between the substrate 52A or 52B or 52C and the lid.

Different substrates 52A, 52A′ and 52B, 52B′ and 52C, 52C′ are shown in FIG. 11A and FIGS. 11B and 11C.

In the example shown in FIG. 11A, the substrates 52A, 52A′ are single layered structures. Examples of suitable single layered structures for the substrate 52A, 52A′ include epoxy siloxane, glass, modified or functionalized glass, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon (polyamides), ceramics/ceramic oxides, silica, fused silica, or silica-based materials, aluminum silicate, silicon and modified silicon (e.g., boron doped p+ silicon), silicon nitride (Si₃N₄), silicon oxide (SiO₂), tantalum pentoxide (Ta₂O₅) or other tantalum oxide(s) (TaOₓ), hafnium oxide (HfO₂), carbon, metals, inorganic glasses, or the like.

In the examples shown in FIG. 11B and FIG. 11C, the substrates 52B, 52B′ and 52C, 52C′ are multi-layered structures. The multi-layered structures of the substrates 52B, 52B′ and 52C, 52C′ include a base support 66, 66′ and a patterned material 64 or 64′ on the base support 66, 66′.

The base support 66, 66′ may be any of the examples set forth herein for the single layered structure of the substrate 52A, 52A′.

The patterned material 64 or 64′ may be any material that is capable of being patterned with posts 70, 70′ (FIG. 11B) or depressions 72, 72′ (FIG. 11C).

In an example, the patterned material 64, 64′ may be an inorganic oxide that is selectively applied to the base support 66, 66′, e.g., via vapor deposition, aerosol printing, or inkjet printing, in the desired pattern. Examples of suitable inorganic oxides include tantalum oxide (e.g., Ta₂O₅), aluminum oxide (e.g., Al₂O₃), silicon oxide (e.g., SiO₂), hafnium oxide (e.g., HfO₂), etc.

In another example, the patterned material 64, 64′ may be a resin matrix material that is applied to the base support 66, 66′ and then patterned. Suitable deposition techniques include chemical vapor deposition, dip coating, dunk coating, spin coating, spray coating, puddle dispensing, ultrasonic spray coating, doctor blade coating, aerosol printing, screen printing, microcontact printing, etc. Suitable patterning techniques include photolithography, nanoimprint lithography (NIL), stamping techniques, embossing techniques, molding techniques, microetching techniques, printing techniques, etc. Some examples of suitable resins include a polyhedral oligomeric silsesquioxane-based resin, a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

As used herein, the term “polyhedral oligomeric silsesquioxane” (commercially available under the tradename POSS® from Hybrid Platics) refers to a chemical composition that is a hybrid intermediate (e.g., RSiO_{1.5}) between that of silica (SiO₂) and silicone (R₂SiO). An example of polyhedral oligomeric silsesquioxane can be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for polyhedral oligomeric silsesquioxane include epoxy, azide/azido, a thiol, a poly(ethylene glycol), a norbornene, a tetrazine, acrylates, and/or methacrylates, or further, for example, alkyl, aryl, alkoxy, and/or haloalkyl groups. The resin composition disclosed herein may comprise one or more different cage or core structures as monomeric units. The average cage content can be adjusted during the synthesis, and/or controlled by purification methods, and a distribution of cage sizes of the monomeric unit(s) may be used in the examples disclosed herein.

In an example, the substrates 52A, 52A′ or 52B, 52B′ or 52C, 52C′ (whether single or multi-layered) may be round and have a diameter ranging from about 2 mm to about 300 mm, or may be a rectangular sheet or panel having its largest dimension up to about 10 feet (˜3 meters). In an example, the substrate 52A, 52A′ or 52B, 52B′ or 52C, 52C′ is a wafer having a diameter ranging from about 200 mm to about 300 mm. Wafers may subsequently be diced to form an individual flow cell substrate. In another example, the substrate 52A, 52A′ or 52B, 52B′ or 52C, 52C′ is a die having a width ranging from about 0.1 mm to about 10 mm. While example dimensions have been provided, it is to be understood that a substrate 52A, 52A′ or 52B, 52B′ or 52C, 52C′ with any suitable dimensions may be used. For another example, a panel may be used that is a rectangular support, which has a greater surface area than a 300 mm round wafer. Panels may subsequently be diced to form individual flow cells.

The flow cell 50 also includes the flow channel 54. While several flow channels 54 are shown in FIG. 10, it is to be understood that any number of flow channels 54 may be included in the flow cell 50 (e.g., a single channel 54, four channels 54, etc.). Each flow channel 54 may be isolated from each other flow channel 54 in a flow cell 50 so that fluid introduced into any particular flow channel 54 does not flow into any adjacent flow channel 54.

A portion of the flow channel 54 may be defined in the substrate 52A, 52A′ or 52B, 52B′ or 52C, 52C′ using any suitable technique that depends, in part, upon the material(s) of the substrate 52A, 52A′ or 52B, 52B′ or 52C, 52C′. In one example, a portion of the flow channel 54 is etched into a glass substrate, such as substrate 52A, 52A′. In another example, a portion of the flow channel 54 may be patterned into a resin matrix material of a multi-layered structure using photolithography, nanoimprint lithography, etc. A separate material (e.g., material 62 in FIG. 11A, FIG. 11B, and FIG. 11C may be applied to the substrate 52A, 52A′ or 52B, 52B′ or 52C, 52C′ so that the separate material 62 defines at least a portion of the walls of the flow channel 54.

In an example, the flow channel 54 has a substantially rectangular configuration with rounded ends. The length and width of the flow channel 54 may be smaller, respectively, than the length and width of the substrate 52A, 52A′ or 52B, 52B′ or 52C, 52C′ so that a portion of the substrate surface surrounding the flow channel 54 is available for attachment to another substrate 52A, 52A′ or 52B, 52B′ or 52C, 52C′ or to a lid. In some instances, the width of each flow channel 54 can be at least about 1 mm, at least about 2.5 mm, at least about 5 mm, at least about 7 mm, at least about 10 mm, or more. In some instances, the length of each flow channel 54 can be at least about 10 mm, at least about 25 mm, at least about 50 mm, at least about 100 mm, or more. The width and/or length of each flow channel 54 can be greater than, less than or between the values specified above. In another example, the flow channel 54 is square (e.g., 10 mm×10 mm).

The depth of each flow channel 54 can be as small as a few monolayers thick, for example, when microcontact, aerosol, or inkjet printing is used to deposit the separate material 62 that defines the flow channel walls. In other examples, the depth of each flow channel 54 can be about 1 μm, about 10 μm, about 50 μm, about 100 μm, or more. In an example, the depth may range from about 10 μm to about 100 μm. In another example, the depth is about 5 μm or less. It is to be understood that the depth of each flow channel 54 can also be greater than, less than or between the values specified above. The depth of the flow channel 54 may also vary along the length and width of the flow cell 50, e.g., when posts 70, 70′ or depressions 72, 72′ are used.

In the example shown in FIG. 11A, each substrate 52A, 52A′ has a substantially flat surface 58, 58′; and the plurality of capture sites 60, 60′ are positioned in a pattern across the substantially flat surfaces 58, 58′.

The substantially flat surfaces 58, 58′ may be the bottom surface of lanes 56, 56′ that are defined in the single layer substrate 52A, 52A′. A lane 56, 56′ may also be defined in the patterned layer 64, 64′ of a multi-layered substrate 52B, 52B′, 52C, 52C′. The lanes 56, 56′ may be etched into the substrate or defined, e.g., by lithography or another suitable technique.

The plurality of capture sites 60, 60′ are positioned in a pattern across the substantially flat surface 58, 58′.

Many different patterns for the capture sites 60, 60′ may be envisaged, including regular, repeating, and non-regular patterns. In an example, the capture sites 60, 60′ are disposed in a hexagonal grid for close packing and improved density. Other layouts may include, for example, rectilinear (rectangular) layouts, triangular layouts, and so forth. In some examples, the layout or pattern can be an x-y format of capture sites 60, 60′ that are in rows and columns. In some other examples, the layout or pattern can be a repeating arrangement of capture sites 60, 60′ separated by regions of the substantially flat substrate 58, 58′. In still other examples, the layout or pattern can be a random arrangement of capture sites 60, 60′. The pattern may include stripes, swirls, lines, triangles, rectangles, circles, arcs, checks, diagonals, arrows, and/or squares.

The layout or pattern of the capture sites 60, 60′ may be characterized with respect to the density of the capture sites 60, 60′ (e.g., number of capture sites 60, 60′) in a defined area. For example, the capture sites 60, 60′ may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density of capture sites 60, 60′ can be between one of the lower values and one of the upper values selected from the ranges above. As examples, a high density array may be characterized as having capture sites 60, 60′ separated by less than about 100 nm, a medium density array may be characterized as having capture sites 60, 60′ separated by about 400 nm to about 1 μm, and a low density array may be characterized as having capture sites 60, 60′ separated by greater than about 1 μm. While example densities have been provided, it is to be understood that any suitable densities may be used. In some instances, it may be desirable for the spacing between capture sites 60, 60′ to be even greater than the examples listed herein.

The layout or pattern of the capture sites 60, 60′ may also or alternatively be characterized in terms of the average pitch, or the spacing from the center of one capture site 60, 60′ to the center of an adjacent capture site 60, 60′ (center-to-center spacing) or from the left edge of one capture site 60, 60′ to the right edge of an adjacent capture site 60, 60′ (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern of capture sites 60, 60′ can be between one of the lower values and one of the upper values selected from the ranges above. In an example, the capture sites 60, 60′ have a pitch (center-to-center spacing) of about 1.5 μm. While example average pitch values have been provided, it is to be understood that other average pitch values may be used.

The capture sites 60, 60′ may have any suitable shape, geometry and dimensions, which may depend, at least in part, on the functionalized nanostructure 10A, 10B, 10C, 10D that is to be captured by the capture site 60, 60′.

The capture sites 60, 60′ may be chemical capture sites, electrostatic captures sites, or magnetic capture sites.

Chemical capture sites include any example of the chemical capture agent set forth herein that can be deposited on or otherwise attached to predefined locations of the substantially flat surface 58, 58′. In one example, the chemical capture agent may be deposited, e.g., using microcontact printing, aerosol printing, etc., in a desirable location on the substantially flat surface 58, 58′ to form the capture sites 60, 60′. In another example, a mask (e.g., a photoresist) may be used to define the space/location where the chemical capture agent will be deposited. The chemical capture agent may then be deposited, and the mask removed (e.g., via lift-off, dissolution, or another suitable technique). In this example, the chemical capture agent may form a monolayer or thin layer of the chemical capture agent. In still another example, a polymer grafted with capture nucleic acids may be selectively applied to the substantially flat surface 58, 58′ to form the chemical captures sites.

Electrostatic captures sites include any example of the electrostatic capture agents set forth herein that can be deposited on predefined locations of the substantially flat surface 58, 58′. For example, electrode materials may be deposited using chemical vapor deposition, masking and deposition, or another suitable technique to form the capture sites 60, 60′. When electrostatic capture sites are used, the substrate 52A, 52A′ may include additional circuitry to address the individual capture sites 60, 60′.

Magnetic capture sites include any example of the magnetic capture agent set forth herein that can be deposited on predefined locations of the substantially flat surface 58, 58′. For example, magnetic materials may be deposited using chemical vapor deposition, masking and deposition, or another suitable technique to form the capture sites 60, 60′.

In the example of FIG. 11A, areas of the substantially flat surface 58, 58′ that do not contain the capture sites 60, 60′ function as interstitial regions between the capture sites 60, 60′.

In the example shown in FIG. 11B, the substrate 52B, 52B′ includes posts 70, 70′ separated by interstitial regions 68, 68′; and a capture site 60, 60′ is positioned over each of the posts 70, 70′.

Each post 70, 70′ is a three-dimensional structure that extends outward (upward) from an adjacent surface. The post 70, 70′ is thus a convex region with respect to the interstitial regions 68, 68′ that surround the posts 70, 70′. Posts 70, 70′ may be formed in or on a substrate 52B, 52B′. In FIG. 11B, the posts 70, 70′ are formed in the substrate 52B, 52B′. When the post 70, 70′ is formed “in the substrate”, it is meant that the layer 64, 64′ is patterned (e.g., via etching, photolithography, imprinting, etc.,) so that the resulting posts 70, 70′ extend above the adjacent surrounding interstitial regions 68, 68′. Alternatively, when the post 70, 70′ is formed “on the substrate”, it is meant that an additional material may be deposited on the substrate (e.g., on the single layer substrate) so that it extends above the underlying substrate.

The layout or pattern of the posts 70, 70′ may be any of the examples set forth herein for the capture sites 60, 60′. The layout or pattern of the posts 70, 70′ may be characterized with respect to the density of the posts 70, 70′ (e.g., number of posts 70, 70′) in a defined area. Any of the densities set forth for the capture sites 60, 60′ may be used for the posts 70, 70′. The layout or pattern of the posts 70, 70′ may also be characterized in terms of the average pitch, or the spacing from the center of one post 70, 70′ to the center of an adjacent post 70, 70′ (center-to-center spacing) or from the left edge of one post 70, 70′ to the right edge of an adjacent post 70, 70′ (edge-to-edge spacing). Any of the average pitches set forth for the capture sites 60, 60′ may be used for the posts 70, 70′.

While any suitable three-dimensional geometry may be used for the posts 70, 70′, a geometry with an at least substantially flat top surface may be desirable so that the capture site 60, 60′ may be formed thereon. Example post geometries include a sphere, a cylinder, a cube, polygonal prisms (e.g., rectangular prisms, hexagonal prisms, etc.), or the like.

The size of each post 70, 70′ may also be characterized by its top surface area, height, and/or diameter.

The top surface area of each post 70, 70′ can be selected based upon the size of the functionalized nanostructure 10A, 10B, 10C, 10D that is to be anchored to the capture site 60, 60′ that is supported by the post 70, 70′. For example, the top surface area of each post 70, 70′ can be at least about 1×10⁻⁴ μm², at least about 1×10⁻³ μm², at least about 0.1 μm², at least about 1 μm², at least about 10 μm², at least about 100 μm², or more. Alternatively or additionally, the top surface area of each post 70, 70′ can be at most about 1×10⁴ μm², at most about 100 μm², at most about 10 μm², at most about 1 μm², at most about 0.1 μm², at most about 1×10⁻² μm², or less. The area occupied by each post top surface can be greater than, less than or between the values specified above.

The height of each post 70, 70′ can depend upon the channel 54 dimensions. In an example, the height may be at least about 0.1 μm, at least about 0.5 μm, at least about 1 μm, at least about 10 μm, at least about 100 μm, or more. Alternatively or additionally, the height can be at most about 1×10³ μm, at most about 100 μm, at most about 10 μm, or less. In some examples, the depth is about 0.4 μm. The height of each post 70, 70′ can be greater than, less than or between the values specified above.

In some instances, the diameter or length and width of each post 70, 70′ can be at least about 50 nm, at least about 0.1 μm, at least about 0.5 μm, at least about 1 μm, at least about 10 μm, at least about 100 μm, or more. Alternatively or additionally, the diameter or length and width can be at most about 1×10³ μm, at most about 100 μm, at most about 10 μm, at most about 1 μm, at most about 0.5 μm, at most about 0.1 μm, or less (e.g., about 50 nm). In some examples, the diameter or length and width is about 0.4 μm. The diameter or length and width of each post 70, 70′ can be greater than, less than or between the values specified above.

In the example shown in FIG. 11B, a respective capture site 60, 60′ is positioned on each of the posts 70, 70′. The capture sites 60, 60′ may be chemical capture sites, electrostatic captures sites, or magnetic capture sites.

Chemical capture sites include any example of the chemical capture agent set forth herein that can be deposited on or otherwise attached to the top surface of each post 70, 70′. In one example, the chemical capture agent may be deposited, e.g., using microcontact printing, aerosol printing, etc., on each post 70, 70′ to form the capture site 60, 60′. In another example, a mask (e.g., a photoresist) may be used to cover the interstitial regions 68, 68′ and not the posts 70, 70′. The chemical capture agent may then be deposited on the exposed posts 70, 70′, and the mask removed (e.g., via lift-off, dissolution, or another suitable technique). In this example, the chemical capture agent may form a monolayer or thin layer of the chemical capture agent on the post 70, 70′. In still another example, a polymer grafted with capture nucleic acids may be selectively applied to the top surface of each post 70, 70′ to form the chemical captures sites.

Electrostatic captures sites include any example of the electrostatic capture agent set forth herein that can be deposited on the top surface of each post 70, 70′. For example, electrode materials may be deposited using chemical vapor deposition, masking and deposition, or another suitable technique to form the capture sites 60, 60′. When electrostatic capture sites are used, the substrate 52B, 52B′ may include additional circuitry to address the individual capture sites 60, 60′.

Magnetic capture sites include any example of the magnetic capture agent set forth herein that can be deposited on the top surface of each post 70, 70′. For example, magnetic materials may be deposited using chemical vapor deposition, masking and deposition, or another suitable technique to form the capture sites 60, 60′.

In the example shown in FIG. 11C, the substrate 52C, 52C′ includes depressions 72, 72′ separated by interstitial regions 68, 68′; and a capture site 60, 60′ is positioned in each of the depressions 72, 72′.

Each depression 72, 72′ is a three-dimensional structure that extends inward (downward) from an adjacent surface. The depression 72, 72′ is thus a concave region with respect to the interstitial regions 42′ that surround the depressions 72, 72′. Depressions 72, 72′ may be formed in a substrate 52C, 52C′. In the example shown in FIG. 11C, the layer 64, 64′ is patterned (e.g., via etching, photolithography, imprinting, etc.,) to define the depressions 72, 72′ so that the interstitial regions 68, 68′ extend above and surround the adjacent depressions 72, 72′.

The layout or pattern of the depressions 72, 72′ may be any of the examples set forth herein for the capture sites 60, 60′. The layout or pattern of the depressions 72, 72′ may be characterized with respect to the density of the depressions 72, 72′ (e.g., number of depressions 72, 72′) in a defined area. Any of the densities set forth for the capture sites 60, 60′ may be used for the depressions 72, 72′. The layout or pattern of the depressions 72, 72′ may also be characterized in terms of the average pitch, or the spacing from the center of one depression 72, 72′ to the center of an adjacent depression 72, 72′ (center-to-center spacing) or from the left edge of one depression 72, 72′ to the right edge of an adjacent depression 72, 72′ (edge-to-edge spacing). Any of the average pitches set forth for the capture sites 60, 60′ may be used for the depressions 72, 72′.

While any suitable three-dimensional geometry may be used for the depressions 72, 72′, a geometry with an at least substantially flat bottom surface may be desirable so that the capture site 60, 60′ may be formed thereon. Example depression geometries include a sphere, a cylinder, a cube, polygonal prisms (e.g., rectangular prisms, hexagonal prisms, etc.), or the like.

The size of each depression 72, 72′ may be characterized by its volume, opening area, depth, and/or diameter.

Each depression 72, 72′ can have any volume that is capable of receiving the material of the capture site 60, 60′. For example, the volume can be at least about 1×10⁻³ μm³, at least about 1×10⁻² μm³, at least about 0.1 μm³, at least about 1 μm³, at least about 10 μm³, at least about 100 μm³, or more. Alternatively or additionally, the volume can be at most about 1×10⁴ μm³, at most about 1×10³ μm³, at most about 100 μm³, at most about 10 μm³, at most about 1 μm³, at most about 0.1 μm³, or less.

The area occupied by each depression opening can be selected based on the size of the functionalized nanostructures 10A, 10B, 10C, 10D to be anchored by the capture site 60, 60′. It may be desirable for the functionalized nanostructures 10A, 10B, 10C, 10D to enter the depression 72, 72′, and thus the area occupied by the depression opening may be bigger than the size of the functionalized nanostructures 10A, 10B, 10C, 10D. For example, the area for each depression opening can be at least about 1×10⁻³ μm², at least about 1×10⁻² μm², at least about 0.1 μm², at least about 1 μm², at least about 10 μm², at least about 100 μm², or more. Alternatively or additionally, the area can be at most about 1×10³ μm², at most about 100 μm², at most about 10 μm², at most about 1 μm², at most about 0.1 μm², at most about 1×10⁻² μm², or less. The area occupied by each depression opening can be greater than, less than or between the values specified above.

The depth of each depression 72, 72′ is large enough to house at least the capture site 60, 60′. In one example, the depression 72, 72′ may be filled with the capture site 60, 60′. In this example, the functionalized nanostructure 10A, 10B, 10C, 10D becomes anchored to the capture site 60, 60′ but does not enter the depression 72, 72′. In another example, the depression 72, 72′ may be partially filled with the capture site 60, 60′. In this example, the functionalized nanostructure 10A, 10B, 10C, 10D at least partially enters the depression 72, 72′ and becomes anchored to the capture site 60, 60′ in the depression 72, 72′. In an example, the depth may be at least about 0.1 μm, at least about 0.5 μm, at least about 1 μm, at least about 10 μm, at least about 100 μm, or more. Alternatively or additionally, the depth can be at most about 1×10³ μm, at most about 100 μm, at most about 10 μm, or less. In some examples, the depth is about 0.4 μm. The depth of each depression 72, 72′ can be greater than, less than or between the values specified above.

In some instances, the diameter or length and width of each depression 72, 72′ can be at least about 50 nm, at least about 0.1 μm, at least about 0.5 μm, at least about 1 μm, at least about 10 μm, at least about 100 μm, or more. Alternatively or additionally, the diameter or length and width can be at most about 1×10³ μm, at most about 100 μm, at most about 10 μm, at most about 1 μm, at most about 0.5 μm, at most about 0.1 μm, or less (e.g., about 50 nm). In some examples, the diameter or length and width is about 0.4 μm. The diameter or length and width of each depression 72, 72′ can be greater than, less than or between the values specified above.

In the example shown in FIG. 11C, the capture site 60, 60′ is positioned in each of the depressions 72, 72′. The capture sites 60, 60′ may be chemical capture sites, electrostatic captures sites, or magnetic capture sites.

Chemical capture sites include any example of the chemical capture agent set forth herein that can be deposited on or otherwise attached to the bottom surface of each depression 72, 72′. In one example, the chemical capture agent may be deposited, e.g., using microcontact printing, aerosol printing, etc., on each depression 72, 72′ to form the capture sites 60, 60′. In another example, a mask (e.g., a photoresist) may be used to cover the interstitial regions 68, 68′ and not the depressions 72, 72′. The chemical capture agent may then be deposited in the exposed depression 72, 72′, and the mask removed (e.g., via lift-off, dissolution, or another suitable technique). In this example, the chemical capture agent may form a monolayer or thin layer of the chemical capture agent in the depression 72, 72′. In still another example, a polymer grafted with capture nucleic acids may be selectively applied to the bottom surface of each depression 72, 72′.

Electrostatic captures sites include any example of the electrostatic capture agent set forth herein that can be deposited on the bottom surface of each depression 72, 72′. For example, electrode materials may be deposited using chemical vapor deposition, masking and deposition, or another suitable technique to form the capture sites 60, 60′. When electrostatic capture sites are used, the substrate 52C, 52C′ may include additional circuitry to address the individual capture sites 60, 60′.

Magnetic capture sites include any example of the magnetic capture agent set forth herein that can be deposited on the bottom surface of each depression 72, 72′. For example, magnetic materials may be deposited using chemical vapor deposition, masking and deposition, or another suitable technique to form the capture sites 60, 60′.

While the example architectures shown in FIG. 11A, FIG. 11B, and FIG. 11C depict the functionalized nanostructures 10A, 10B, 10C, 10D anchored at the captures sites 60, 60′, it is to be understood that the flow cell 50 does not include the functionalized nanostructures 10A, 10B, 10C, 10D until they are introduced thereto, e.g., during sequencing.

Flow Cells including Functionalized Transparent Metal Films

As mentioned herein, the spatially separated functionalized primer sets 30, 32 may be attached to the transparent metal film 74 instead of the metal nanostructures 22. This example may be particular desirable for use in the flow cell 50′ shown in FIG. 12, which includes a complementary metal oxide semiconductor chip 94.

In addition to the complementary metal oxide semiconductor chip 94, this example flow cell 50′ includes a substrate 52D over the complementary metal oxide semiconductor chip 94, the substrate 52D including a plurality of depressions 72 separated by interstitial regions 68; the transparent metal film 74 positioned on a bottom surface in each of the plurality of depressions 72; an un-cleavable first primer 34 and a cleavable second primer 36 attached to a first region 74A of the transparent metal film 74 through i) a first thiol linkage 16 attached to a first polymer chain 12B, 12C, 12D having a first polarity or ii) respective first thiol linkages 16 attached to respective first polymer chains 12A having the first polarity; and a cleavable first primer 38 and an un-cleavable second primer 40 attached to a second region 74B of the transparent metal film 74 through i) a second thiol linkage 16′ attached to a second polymer chain 14B, 14C, 14D having a second polarity or ii) respective second thiol linkages 16 attached to respective second polymer chains 14A having the second polarity.

As described herein, any of the methods may be adapted so that the functionalized primer sets 30, 32 (i.e., primer-containing polymer chains 18A-18D and 20A-20D) are attached to the transparent metal film 74 in the flow cell depressions 72, 72′.

In the illustrated example, the substrate 52D may be affixed directly to, and thus be in physical contact with, the complementary metal oxide semiconductor chip 94 through one or more securing mechanisms (e.g., adhesive, bond, fasteners, and the like). It is to be understood that the substrate 52D may be removably coupled to the complementary metal oxide semiconductor (CMOS) chip 94.

The CMOS chip 94 includes a plurality of stacked layers 96 including, for example, silicon layer(s), dielectric layer(s), metal-dielectric layer(s), metal layer(s), etc.). The stacked layers 96 make up the device circuitry, which includes detection circuitry.

The CMOS chip 94 includes optical components, such as optical sensor(s) 98 and optical waveguide(s) 100. The optical components are arranged such that each optical sensor 98 at least substantially aligns with, and thus is operatively associated with, a single optical waveguide 100 and a single reaction site 102 of the flow cell 50′. However, in other examples, a single optical sensor 98 may receive photons through more than one optical waveguide 100 and/or from more than one reaction site 102. In these other examples, the single optical sensor 98 is operatively associated with more than one optical waveguide 100 and/or more than one reaction site 102.

As used herein, a single optical sensor 98 may be a light sensor that includes one pixel or more than one pixel. As an example, each optical sensor 98 may have a detection area that is less than about 50 μm². As another example, the detection area may be less than about 10 μm². As still another example, the detection area may be less than about 2 μm². In the latter example, the optical sensor 98 may constitute a single pixel. An average read noise of each pixel of the optical sensor 98 may be, for example, less than about 150 electrons. In other examples, the read noise may be less than about 5 electrons. The resolution of the optical sensor(s) 98 may be greater than about 0.5 megapixels (Mpixels). In other examples, the resolution may be greater than about 5 Mpixels, or greater than about 10 Mpixels.

Also as used herein, a single optical waveguide 100 may be a light guide including a cured filter material that i) filters the excitation light 104 (propagating from an exterior of the flow cell 50′ into the flow channel 54), and ii) permits the light emissions (not shown, resulting from reactions at the reaction site 102) to propagate therethrough toward corresponding optical sensor(s) 98. In an example, the optical waveguide 100 may be, for example, an organic absorption filter. As a specific example, the organic absorption filter may filter excitation light 104 of about 532 nm wavelength and permit light emissions of about 570 nm or more wavelengths. The optical waveguide 100 may be formed by first forming a guide cavity in a dielectric layer 106, and then filling the guide cavity with a suitable filter material.

The optical waveguide 100 may be configured relative to the dielectric material 106 in order to form a light-guiding structure. For example, the optical waveguide 100 may have a refractive index of about 2.0 so that the light emissions are substantially reflected at an interface between the optical waveguide 100 and the surrounding dielectric material 106. In certain examples, the optical waveguide 100 is selected such that the optical density (OD) or absorbance of the excitation light 104 is at least about 4 OD. More specifically, the filter material may be selected and the optical waveguide 100 may be dimensioned to achieve at least 4 OD. In other examples, the optical waveguide 100 may be configured to achieve at least about 5 OD or at least about 6 OD.

In this example, the substrate 52D may be a passivation layer. At least a portion of the substrate 52D (and thus the passivation layer) is in contact with a first embedded metal layer 112 of the CMOS chip 94 and also with an input region 110 of the optical waveguide 100. The contact between the substrate 52D and the first embedded metal layer 112 may be direct contact or may be indirect contact through a shield layer 114.

The substrate 52D (passivation layer) may provide one level of corrosion protection for the embedded metal layer 112 of the CMOS chip 94 that is closest in proximity to the substrate 52D. In this example, the substrate 52D may include a passivation material that is transparent to the light emissions resulting from reactions at the reaction site 102 (e.g., visible light), and that is at least initially resistant to the fluidic environment and moisture that may be introduced into or present in the flow channel 54. An at least initially resistant material acts as an etch barrier to high pH reagents (e.g., pH ranging from 8 to 14) and as a moisture barrier. Examples of suitable materials for the substrate 52D include silicon nitride (Si₃N₄), silicon oxide (SiO₂), tantalum pentoxide (TaO₅), hafnium oxide (HfO₂), boron doped p+ silicon, or the like. The thickness of the substrate 52D may vary depending, in part upon the sensor dimensions. In an example, the thickness of the substrate 52D ranges from about 100 nm to about 500 nm.

The flow cell 50′ also includes a lid 116 that is operatively connected to the substrate 52D to partially define the flow channel 54 between the substrate 52D (and the reaction site(s) 102 therein or thereon) and the lid 116. The lid 116 may be any material that is transparent to the excitation light 104 that is directed toward the reaction site(s) 102. As examples, the lid 116 may include glass (e.g., borosilicate, fused silica, etc.), plastic, etc. A commercially available example of a suitable borosilicate glass is D 263®, available from Schott North America Inc. Commercially available examples of suitable plastic materials, namely cyclo olefin polymers, are the ZEONOR® products available from Zeon Chemicals L.P.

The lid 116 may be physically connected to the substrate 52D through material 62. The material 62 is/are coupled to a portion the surface of the substrate 52D, and extends between that surface and an interior surface of the lid 116. In some examples, the material 62 and the lid 116 may be integrally formed such that they 62, 116 are a continuous piece of material (e.g., glass or plastic). In other examples, the material 62 and the lid 116 may be separate components that are coupled to each other. In these other examples, the material 62 may be the same material as, or a different material than the lid 116. In some of these other examples, at least one of the materials 62 includes an electrode material. In still other examples, the material 62 includes a curable adhesive layer that bonds the lid 116 to the substrate 52D (at a portion of its surface).

In an example, the lid 116 may be a substantially rectangular block having an at least substantially planar exterior surface 118 and an at least substantially planar interior surface 120 that defines a portion of the flow channel 54. The block may be mounted onto the material 62. Alternatively, the block may be etched to define the lid 116 and the material 62 (which function as sidewall(s)). For example, a recess may be etched into the transparent block. When the etched block is mounted to the substrate 52D, the recess may become the flow channel 54.

The lid 116 may include inlet and outlet ports 122, 124 that are configured to fluidically engage other ports (not shown) for directing fluid(s) into the flow channel 54 (e.g., from a reagent cartridge or other fluid storage system component) and out of the flow channel 54 (e.g., to a waste removal system).

The flow channel 54 may be sized and shaped to direct a fluid along the reaction site(s) 102. The height of the flow channel 54 and other dimensions of the flow channel 54 may be configured to maintain a substantially even flow of the fluid along the reaction site(s) 102. The dimensions of the flow channel 54 may also be configured to control bubble formation. In an example, the height of the flow channel 54 may range from about 50 μm to about 400 μm. In another example, the height of the flow channel 54 may range from about 80 μm to about 200 μm. It is to be understood that the height of the flow channel 54 may vary, and may be the greatest when the reaction site 102 is located in a reaction chamber (e.g., depression 72) that is defined in the surface of the substrate 52D. In these examples, the depression 72 increases the height of the flow channel 54 at this particular area.

Each reaction site 102 is a localized region in the substrate 52D where a designated reaction may occur. Each reaction site 102 is a depression 72 having the transparent metal film 74 therein, where the transparent metal film includes regions 74A, 74B functionalized with the primer-containing polymer chains 12A, 12B, 12C, or 12D and 14A, 14B, 14C, and 14D respectively attached thereto.

The metal film 74 may be selectively deposited (e.g., using sputter coating, thermal evaporation, or any other suitable technique) into the depressions 72. The metal film 74 has a thickness ranging from about 1 nm to about 20 nm, and thus the metal film 74 is transparent to the wavelengths used during the sequencing operation.

While not shown in FIG. 12, it is to be understood that the transparent metal film 74 may alternatively be embedded as a layer within the substrate 52D, as long as it is exposed at each of the depressions 72. In this example, the transparent metal film 74 may be sandwiched between two portions of the substrate 52D, except at the depression 72, where a portion of the substrate 52D is removed to expose the transparent metal film 74.

In an example, the reaction site 102 is at least substantially aligned with the input region 110 of a single optical waveguide 100. As such, light emissions at the reaction site 102 may be directed into the input region 110, through the waveguide 100, and to an associated optical sensor 98. In other examples, one reaction site 102 may be aligned with several input regions 110 of several optical waveguides 100. In still other examples, several reaction sites 102 may be aligned with one input region 110 of one optical waveguide 100.

In the examples disclosed herein, the reaction sites 102 may include biological or chemical substances that emit optical (e.g., light) signals. For example, the biological or chemical substances of the reactions sites 102 may generate light emissions in response to the excitation light 104. In particular examples, after library template amplification and clustering, the reaction sites 102 include a cluster of forward library template strands attached to the region 74A and a cluster of reverse library template strands attached to the other region 74B.

The embedded metal layer 112 may be any suitable CMOS metal, such as aluminum (Al), aluminum chloride (AlCu), tungsten (W), nickel (Ni), or copper (Cu). The embedded metal layer 112 is a functioning part of the CMOS AVdd line, and through the stacked layers 96, is also electrically connected to the optical sensor 98. Thus, the embedded metal layer 112 participates in the detection/sensing operation.

It is to be understood that the other optical sensors 98 and associated components may be configured in an identical or similar manner. It is also to be understood, however, that the CMOS chip 94 may not be manufactured identically or uniformly throughout. Instead, one or more optical sensor 98 and/or associated components may be manufactured differently or have different relationships with respect to one another

The stacked layer 96 may include interconnected conductive elements (e.g., conductors, traces, vias, interconnects, etc.) that can conduct electrical current. The circuitry may be configured for selectively transmitting data signals that are based on detected photons. The circuitry may also be configured for signal amplification, digitization, storage, and/or processing. The circuitry may collect and analyze the detected light emissions and generate data signals for communicating detection data to a bioassay system. The circuitry may also perform additional analog and/or digital signal processing in the CMOS chip 94.

The CMOS chip 94 may be manufactured using integrated circuit manufacturing processes. The CMOS chip 94 may include multiple layers, such as a sensor base/layer (e.g., a silicon layer or wafer). The sensor base may include the optical sensor 98. When the CMOS chip 94 is fully formed, the optical sensor 98 may be electrically coupled to the rest of the circuitry in the stack layer 96 through gate(s), transistor(s), etc.

As used in reference to FIG. 12, the term “layer” is not limited to a single continuous body of material unless otherwise noted. For example, the sensor base/layer may include multiple sub-layers that are different materials and/or may include coatings, adhesives, and the like. Furthermore, one or more of the layers (or sub-layers) may be modified (e.g., etched, deposited with material, etc.) to provide the features described herein.

The stacked layer 96 also includes a plurality of metal-dielectric layers. Each of these layers includes metallic elements (e.g., M1-M5, which may be, for example, W (tungsten), Cu (copper), Al (aluminum), or any other suitable CMOS conductive material) and dielectric material 106 (e.g., SiO₂). Various metallic elements M1-M5 and dielectric materials 106 may be used, such as those suitable for integrated circuit manufacturing.

In the example shown in FIG. 12, each of the plurality of metal-dielectric layers L1-L6 includes both metallic elements M1, M2, M3, M4, M5 and dielectric material 106. In each of the layers L1-L6, the metallic elements M1, M2, M3, M4, M5 are interconnected and are embedded within dielectric material 106. In some of the metal-dielectric layers L1-L6, additional metallic elements may also be included. Some of these additional metallic elements may be used to address individual pixels through a row and column selector. The voltages at these elements may vary and switch between about −1.4 V and about 4.4 V depending upon which pixel the device is reading out.

The configuration of the metallic elements M1, M2, M3, M4, M5 and dielectric layer 106 in FIG. 12 is illustrative of the circuitry, and it is to be understood that other examples may include fewer or additional layers and/or may have different configurations of the metallic elements M1-M5.

In the example shown in FIG. 12, the shield layer 114 is in contact with at least a portion of the substrate 52D. The shield layer 114 has an aperture at least partially adjacent to the input region 110 of the optical waveguide 100. This aperture enables the reaction site 102 (and at least some of the light emissions therefrom) to be optically connected to the waveguide 100. It is to be understood that the shield layer 114 may have an aperture at least partially adjacent to the input region 110 of each optical waveguide 100. The shield layer 114 may extend continuously between adjacent apertures.

The shield layer 114 may include any material that can block, reflect, and/or significantly attenuate the light signals that are propagating through the flow channel 54. The light signals may be the excitation light 104 and/or the light emissions from the reaction site(s) 102. As an example, the shield layer 114 may be tungsten (W).

It is to be understood that the flow cell 50′ may also be used for optical detection.

Sequencing Methods

The functionalized nanostructures 10A-10D may be used with any example of the flow cell 50 shown in FIG. 11A through FIG. 11C to perform a sequencing operation. Similarly, the flow cell 50′ may be used to perform a sequencing operation.

One example of the sequencing operation involving the functionalized nanostructures 10A, 10B, 10C, or 10D and the flow cell 50 includes introducing a plurality of functionalized nanostructures 10A, 10B, 10C, or 10D to the flow cell 50, whereby at least some of the plurality of functionalized nanostructures 10A, 10B, 10C, or 10D attach to respective capture sites 60, 60′ on a surface of the flow cell 50; introducing an amplification mix to the flow cell 40, thereby amplifying respective DNA sample strands in the amplification mix using the un-cleavable first primer 34, the cleavable second primer 36, the cleavable first primer 38, and the un-cleavable second primer 40; cleaving amplicons attached to the cleavable first primers 38 and the cleavable second primers 36; and performing a sequencing operation using amplicons attached to the un-cleavable first primers 34 and the un-cleavable second primers 40.

In this particular example, the functionalized nanostructures 10A, 10B, 10C, or 10D are introduced into the flow cell 50 before amplification. In this example, at least some of the functionalized nanostructures 10A, 10B, 10C, or 10D respectively attach to at least some of the capture sites 60, 60′. As described herein, the functionalized nanostructures 10A, 10B, 10C, or 10D include a functional agent, a reversibly chargeable functional group, or magnetic material that specifically binds, attaches, or is otherwise attracted (e.g., electrostatically, magnetically, etc.) to the capture site 60, 60′. A suspension containing the functionalized nanostructures 10A, 10B, 10C, or 10D may be allowed to incubate in the flow cell 50 for a predetermined time to allow the functionalized nanostructures 10A, 10B, 10C, or 10D to become anchored. When electrostatic capture sites 60, 60′ are used, the individual sites 60, 60′ may be electrically addressed to move the functionalized nanostructures 10A, 10B, 10C, or 10D toward individual capture sites 60, 60′. In this example, the functionalized nanostructures 10A, 10B, 10C, or 10D may include a reversibly chargeable functional group that can be converted from a neutral species to a charged species at a suitable pH. The charged species can be generated by adjusting the pH, and then attracted to the electrostatic capture sites 60, 60′ that are individually or globally addressed. A rinse may be performed to remove any non-immobilized functionalized nanostructures 10A, 10B, 10C, or 10D, and amplification may be performed on-board the flow cell 50 as described herein.

In another example of the sequencing operation involving the functionalized nanostructures 10A, 10B, 10C, or 10D and the flow cell 50, amplification takes place off of the flow cell 50. In this example, the functionalized nanostructures 10A, 10B, 10C, or 10D are mixed with the amplification mix outside (i.e., off board) of the flow cell 50. The components may be mixed in a sample tube or other reaction vessel where the amplification process (an example of which is described in more detail below) of the respective DNA sample strands using the un-cleavable first primer 34, the cleavable second primer 36, the cleavable first primer 38, and the un-cleavable second primer 40 takes place. The amplicons attached to the cleavable first primers 38 and the cleavable second primers 36 are cleaved, leaving amplicons attached to the un-cleavable first primers 34 and the un-cleavable second primers 40. This method forms pre-clustered nanostructures. The-pre-clustered nanostructures are then introduced into the flow cell 50 where they attach to the capture sites 60, 60′. The amplicons attached to the un-cleavable first primers 34 and the un-cleavable second primers 40 may then be sequenced.

Still another example of the sequencing operation involves the flow cell 50′. This method includes introducing an amplification mix to the flow cell 50′ (which includes the CMOS chip 94 and the other components described in reference to FIG. 12), thereby amplifying respective DNA sample strands in the amplification mix using the un-cleavable first primer 34, the cleavable second primer 36, the cleavable first primer 38, and the un-cleavable second primer 40; cleaving amplicons attached to the cleavable first primer 38 and the cleavable second primer 36; and performing a sequencing operation using amplicons attached to the un-cleavable first primer 34 and the un-cleavable second primer 40.

In any of these methods, the respective DNA sample strands (i.e., library templates) may be prepared from any nucleic acid sample (e.g., a DNA sample or an RNA sample). The DNA nucleic acid sample may be fragmented into single-stranded, similarly sized (e.g., <1000 bp) DNA fragments. The RNA nucleic acid sample may be used to synthesize complementary DNA (cDNA), and the cDNA may be fragmented into single-stranded, similarly sized (e.g., <1000 bp) cDNA fragments. During preparation, adapters may be added to the ends of any of the fragments. Through reduced cycle amplification, different motifs may be introduced in the adapters, such as sequencing primer binding sites, indices, and regions that are complementary to the primers 34, 36 and 38, 40 on the functionalized nanostructures 10A, 10B, 10C, or 10D. In some examples, the fragments from a single nucleic acid sample have the same adapters added thereto. The final library templates include the DNA or cDNA fragment and adapters at both ends. The DNA or cDNA fragment represents the portion of the final DNA sample strand that is to be sequenced.

When amplification is performed on the flow cell 50 or 50′, the DNA sample strands are added to the flow cell 50 or 50′ in an amplification mix, which includes a liquid carrier and a high-fidelity DNA polymerase. When amplification is performed off of the flow cell 50, the DNA sample strands are introduced to a suspension including the functionalized nanostructures 10A, 10B, 10C, or 10D in a liquid carrier. The high-fidelity DNA polymerase may be added to the suspension for off-board amplification. In either of these examples, the liquid carrier may include a buffer (e.g., a Tris-HCl buffer or 0.5× saline sodium citrate (SSC) buffer), acetic acid, acetone, acetonitrile, benzene, butanol, diethylene glycol, diethyl ether, dimethyl formamide, ethanol, glycerin, methane, pyridine, triethyl amine, etc. Surfactants/dispersants, such as sodium dodecyl sulfate (SDS), (CTAB) may also be included.

During on-flow cell or off-flow cell amplification, multiple DNA sample strands are hybridized, for example, to the primers 34, 38 (which have the same sequence except for the cleavage site 42′ or 44) or 36, 40 (which have the same sequence except for the cleavage site 42) immobilized to the metal surface (nanostructure 22 or metal film 74).

Amplification of the template DNA sample strands may be initiated to form clustered nanostructures with a cluster of the template strands across the regions 22A, 22B or clusters of the template strands across the regions 74A, 74B of the transparent metal film 74. The clustered nanostructures may be formed before or after the functionalized nanostructures 10A, 10B, 10C, 10D are introduced into the flow cell 50 immobilized at the capture sites 60, 60′.

In one example, amplification involves cluster generating. In one example of cluster generation, the DNA sample strands are copied from the hybridized primers by 3′ extension using the high-fidelity DNA polymerase. The original DNA sample strands are denatured, leaving the copies (amplicons) immobilized all around the functionalized nanostructures 10A, 10B, 10C, 10D or the metal film 74. Isothermal bridge amplification or some other form of amplification may be used to amplify the immobilized copies. For example, the copied templates loop over to hybridize to an adjacent, complementary primer, and a polymerase copies the copied templates to form double stranded bridges, which are denatured to form two single stranded strands. These two strands loop over and hybridize to adjacent, complementary primers and are extended again to form two new double stranded loops. The process is repeated on each template copy by cycles of isothermal denaturation and amplification to create dense clonal clusters on the functionalized nanostructures 10A, 10B, 10C, 10D or the metal film 74. Each cluster of double stranded bridges is denatured.

This results in amplicons that are attached to the un-cleavable primers 34, 40 and to the cleavable primers 36, 38. In one region 22A or 74A, forward strands are attached to the un-cleavable primers 34, and in the other region 22B or 74B, reverse strands are attached to the un-cleavable primers 40. In this example, reverse strands are attached to the cleavable primers 36 and forward strands are attached to the cleavable primers 38. Alternatively, in one region 22A or 74A, reverse strands are attached to the un-cleavable primers 34, and in the other region 22B or 74B, forward strands are attached to the un-cleavable primers 40. In this alternate example, forward strands are attached to the cleavable primers 36 and reverse strands are attached to the cleavable primers 38.

This example of clustering is referred to as bridge amplification, and is one example of the amplification that may be performed. It is to be understood that other amplification techniques may be used.

The amplicons attached to the cleavable primers 36, 38 may then be removed by introducing a chemical agent or an enzymatic cleaving agent depending on the cleavage sites 42, 42′ or 42, 44. After cleavage, the amplicons attached to the un-cleavable primers 34, 40 in the respective regions 22A or 74A and 22B or 74B remain.

The pre-clustered nanostructures or the flow channel 54 of the flow cell 50 (containing clusters on the already immobilized nanostructures 10A, 10B, 10C, 10D) or 50′ (containing clusters across the metal film 74) may be washed to remove unreacted template DNA sample strands, etc.

When off-flow cell amplification is performed, the washed and pre-clustered nanostructures may be suspended in a fresh carrier liquid for introduction into the flow cell 50. Upon introduction into the flow cell 50, at least some of the pre-clustered nanostructures will immobilize onto at least some of the capture sites 60, 60′ in a manner specific to the type of mechanism included in the nanostructures 10A, 10B, 10C, 10D. A wash cycle may be performed to remove any unanchored pre-clustered nanostructures.

Sequencing primers may then be introduced to the flow cell 50 or 50′. The sequencing primers hybridize to a complementary portion of the sequence of the amplicons that are attached to the functionalized nanoparticles 10A, 10B, 10C, 10D or to the metal film 74. These sequencing primers render the amplicons ready for sequencing.

An incorporation mix including labeled nucleotides may then be introduced into the flow cell 50 or 50′, e.g., via an inlet port. In addition to the labeled nucleotides, the incorporation mix may include water and/or an ionic salt buffer fluid, such as saline citrate at milli-molar to molar concentrations, sodium chloride, potassium chloride, phosphate buffered saline, etc., and/or other buffers, such as tris(hydroxymethyl)aminomethane (TRIS) or (4-(2-hydroxyethyl)-1-piperazineethanesulfonic acid) (HEPES). The liquid carrier may also include catalytic metal(s) intended for the incorporation reaction, such as Mg²⁺, Mn²⁺, Ca²⁺, etc. A single catalytic metal or a combination of catalytic metals may be used, and the total concentration may range from about 0.01 mM to about 100 mM. The incorporation mix also includes a polymerase that can accept the labeled nucleotides, and that can successfully incorporate the nucleotide base into a nascent strand along an amplicon. Examples polymerases include those polymerases from family A, such as Bsu Polymerase, Bst Polymerase, Taq Polymerase, T7 Polymerase, and many others; polymerases from families B and B2, such as Phi29 polymerase and other highly processive polymerases (family B2), Pfu Polymerase (family B), KOD Polymerase (family B), 9oN (family B), and many others; polymerases from family C, such as Escherichia coli DNA Pol III, and many others, polymerases from family D, such as Pyrococcus furiosus DNA Pol II, and many others; polymerases from family X, such as DNA Pol μ, DNA Pol β, DNA Pol α, and many others.

When the incorporation mix is introduced into the flow cell 50 or 50′, the mix enters the flow channel 54, and contacts the amplicons (on the nanostructures 10A, 10B, 10C, 10D or the metal film 74). The incorporation mix is allowed to incubate in the flow cell 50 or 50′, and labeled nucleotides (including optical labels) are incorporated by respective polymerases into the nascent strands along the amplicons on the nanostructures 10A, 10B, 10C, 10D or the metal film 74.

During incorporation, one of the labeled nucleotides is incorporated, by a respective polymerase, into one nascent strand that extends one sequencing primer and that is complementary to one of the template strands. Incorporation is performed in a template strand dependent fashion, and thus detection of the order and type of labeled nucleotides added to the nascent strand can be used to determine the sequence of the amplicon and thus the original DNA sample stands. Incorporation occurs in at least some of the amplicons across the nanostructures 10A, 10B, 10C, 10D or the metal film 74 during a single sequencing cycle.

The incorporated labeled nucleotides may include a reversible termination property due to the presence of a 3′ OH blocking group, which terminates further sequencing primer extension once the labeled nucleotide has been added. After a desired time for incubation and incorporation, the incorporation mix, including non-incorporated labeled nucleotides, may be removed from the flow cell 20 during a wash cycle. The wash cycle may involve a flow-through technique, where a washing solution (e.g., buffer) is directed into, through, and then out of flow channel 54, e.g., by a pump or other suitable mechanism.

Without further incorporation taking place, the most recently incorporated labeled nucleotides can be detected through an imaging event or a data collection event.

During the imaging event, an illumination system may provide excitation light to the flow cell 50. The optical labels of the incorporated labeled nucleotides emit optical signals in response to the excitation light. An optical imager captures images of the optical signals.

During the data collection event, the illumination system may provide an excitation light (e.g., light 104 in FIG. 12) to the flow cell 50′. Like the imaging event, the optical labels of the incorporated labeled nucleotides emit optical signals in response to the excitation light. The light emissions (e.g., photons) are directed through the waveguide 100 toward the optical sensor 98. The circuitry of the flow cell 50′ collects and analyzes the detected light emissions and generates data signals. The circuitry can communicate/transmit the data signals to a bioassay system. The circuitry may also be configured for signal amplification, digitization, storage, and/or processing. The circuitry may also perform additional analog and/or digital signal processing.

After imaging or data collection is performed, a cleavage mix may then be introduced into the flow cell 50 or 50′. In an example, the cleavage mix is capable of i) removing the 3′ OH blocking group from the incorporated nucleotides, and ii) cleaving the optical label from the incorporated nucleotide. Examples of 3′ OH blocking groups and suitable de-blocking agents/components in the cleavage mix may include: ester moieties that can be removed by base hydrolysis; allyl-moieties that can be removed with NaI, chlorotrimethylsilane and Na₂S₂O₃ or with Hg(II) in acetone/water; azidomethyl which can be cleaved with phosphines, such as tris(2-carboxyethyl)phosphine (TCEP) or tri(hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II); and 3′ phosphate which can be cleaved by phosphatase enzymes (e.g., polynucleotide kinase). Examples of suitable optical label cleaving agents/components in the cleavage mix may include: sodium periodate, which can cleave a vicinal diol; phosphines, such as tris(2-carboxyethyl)phosphine (TCEP) or tris(hydroxypropyl)phosphine (THP), which can cleave azidomethyl linkages; palladium and THP, which can cleave an allyl; bases, which can cleave ester moieties; or any other suitable cleaving agent.

Additional sequencing cycles may then be performed until the amplicons are sequenced.

To further illustrate the present disclosure, an example is given herein. It is to be understood that this example is provided for illustrative purposes and is not to be construed as limiting the scope of the present disclosure.

### NON-LIMITING WORKING EXAMPLE

Acrylamide copolymers of three different molecular weights (10 kDa, 25 kDa, and 50 kDa) were generated using RAFT polymerization. For each of these copolymers, monomers of structure (I) were used with N₃ as R² and monomer of structure (XI) were used with 2 hydrogen R groups, a —C(O)OH R group, and a —C(O)NH₂ R group. The RAFT agent was 4-cyano-4-[(dodecylsulfanylthiocarbonyl)sulfanyl]pentanoic acid. RAFT polymerization took place at about 60° C. for a time ranging from about 2 hours to about 24 hours depending on the desired molecular weight.

The respective copolymers were mixed with gold nanoparticles (80 nm) and were allowed to react. After the reactions took place, the size of the particles was measured by Dynamic Light Scattering (DLS) to determine whether functionalization took place. The results are shown in FIG. 13. The results confirmed that the gold nanoparticles were functionalized with the polymer chains containing end thiol linkages.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

While several examples have been described in detail, it is to be understood that the disclosed examples may be modified. Therefore, the foregoing description is to be considered non-limiting.

1. A functionalized nanostructure, comprising:
a metal nanostructure;
an un-cleavable first primer and a cleavable second primer attached to a first region of the metal nanostructure through i) a first thiol linkage attached to a first polymer chain having a first polarity or ii) respective first thiol linkages attached to respective first polymer chains having the first polarity; and
a cleavable first primer and an un-cleavable second primer attached to a second region of the metal nanostructure through i) a second thiol linkage attached to a second polymer chain having a second polarity different from the first polarity or ii) respective second thiol linkages attached to respective second polymer chains having the second polarity.

2. The functionalized nanostructure as defined in claim 1, wherein the metal nanostructure is selected from the group consisting of gold, platinum, copper, and silver.

3. The functionalized nanostructure as defined in claim 1, wherein:
the un-cleavable first primer and the cleavable second primer are attached to the first region through the respective first thiol linkages attached to the respective first polymer chains;
the un-cleavable first primer and the cleavable second primer are attached to the respective first polymer chains at respective ends that are opposed to the respective first thiol linkages;
the cleavable first primer and the un-cleavable second primer are attached to the second region through the respective second thiol linkages attached to the respective second polymer chains; and
the cleavable first primer and the un-cleavable second primer are attached to the respective second polymer chains at respective ends that are opposed to the respective second thiol linkages.

4. The functionalized nanostructure as defined in claim 3, wherein:
each of the respective first polymer chains is polystyrene; and
each of the respective second polymer chains is poly(ethylene glycol).

5. The functionalized nanostructure as defined in claim 1, wherein:
the un-cleavable first primer and the cleavable second primer are attached to the first region through the first thiol linkage attached to the first polymer chain;
the un-cleavable first primer and the cleavable second primer are attached to respective side chains of the first polymer chain;
the cleavable first primer and the un-cleavable second primer are attached to the second region through the second thiol linkage attached to the second polymer chain; and
the cleavable first primer and the un-cleavable second primer are attached to the respective side chains of the second polymer chain.

6. The functionalized nanostructure as defined in claim 5, wherein:
each of the respective side chains of the first polymer chain is positioned closer to an end of the first polymer chain that is opposed to the first thiol linkage than to the first thiol linkage; and
each of the respective side chains of the second polymer chain is positioned closer to an end of the second polymer chain that is opposed to the second thiol linkage than to the second thiol linkage.

7. The functionalized nanostructure as defined in claim 6, wherein:
a predetermined portion of a backbone of the first polymer chain that is adjacent to the first thiol linkage is free of the respective side chains of the first polymer chain; and
a predetermined portion of a backbone of second first polymer chain that is adjacent to the second thiol linkage is free of the respective side chains of the first polymer chain.

8. The functionalized nanostructure as defined in claim 5, wherein:
the respective side chains of the first polymer chain are distributed along a backbone of the first polymer chain; and
the respective side chains of the second polymer chain are distributed along a backbone of the second polymer chain.

9. A method, comprising:
generating a first functionalized primer set by attaching an un-cleavable first primer and a cleavable second primer to i) a first polymer chain having a first polarity and a first thiol linkage or ii) respective first polymer chains having the first polarity and respective first thiol linkages;
generating a second functionalized primer set by attaching each of a cleavable first primer and an un-cleavable second primer to i) a second polymer chain having a second polarity different from the first polarity and a second thiol linkage or ii) respective second polymer chains having the second polarity and respective second thiol linkages; and
attaching the first and second functionalized primer sets, through the first thiol linkage and the second thiol linkage or the respective first thiol linkages and the respective second thiol linkages, to spatially separate regions of a metal surface.

10. The method as defined in claim 9, wherein:
generating the first functionalized primer set involves reacting respective terminal end functional groups of the un-cleavable first primer and of the cleavable second primer with respective ends groups of the respective first polymer chains; and
generating the second functionalized primer set involves reacting respective terminal end functional groups of the cleavable first primer and of the un-cleavable second primer with respective ends groups of the respective second polymer chains.

11. The method as defined in claim 10, wherein:
the first functionalized primer set and the second functionalized primer set are generated in separate reaction containers; and
the method further comprises:
mixing the first functionalized primer set and the second functionalized primer set to form a mixture; and
exposing the mixture to the metal surface.

12. The method as defined in claim 11, wherein one of:
the metal surface includes metal nanostructures, and exposing the mixture to the metal surface involves adding the metal nanostructures to the mixture; or
the metal surface includes a transparent metal film positioned on a bottom surface in each of a plurality of depressions of a flow cell, and exposing the mixture to the metal surface involves incubating the mixture in each of the plurality of depressions.

13. The method as defined in claim 9, wherein:
generating the first functionalized primer set involves:
polymerizing a first primer grafting monomer and a first backbone monomer to introduce primer reactive side chains to the first polymer chain; and
respectively grafting the un-cleavable first primer and the cleavable second primer to the primer reactive side chains of the first polymer chain; and
generating the second functionalized primer set involves:
polymerizing a second primer grafting monomer and a second backbone monomer to introduce primer reactive side chains to the second polymer chain; and
respectively grafting the cleavable first primer and the un-cleavable second primer to the primer reactive side chains of the second polymer chain.

14. The method as defined in claim 13, wherein:
the first functionalized primer set and the second functionalized primer set are generated in separate reaction containers; and
the method further comprises:
mixing the first functionalized primer set and the second functionalized primer set to form a mixture; and
exposing the mixture to the metal surface.

15. The method as defined in claim 14, wherein one of:
the metal surface includes metal nanostructures, and exposing the mixture to the metal surface involves adding the metal nanostructures to the mixture; or
the metal surface includes a transparent metal film positioned on a bottom surface in each of a plurality of depressions of a flow cell, and exposing the mixture to the metal surface involves incubating the mixture in each of the plurality of depressions.

16. The method as defined in claim 9, wherein:
generating the first functionalized primer set involves:
polymerizing a first primer grafting monomer and a first backbone monomer to form a first portion of the first polymer chain including primer reactive side chains;
continuing polymerization with the first backbone monomer to form a second portion of the first polymer chain without primer reactive side chains; and
respectively grafting the un-cleavable first primer and the cleavable second primer to the primer reactive side chains of the first portion of the first polymer chain; and
generating the second functionalized primer set involves:
polymerizing a second primer grafting monomer and a second backbone monomer to form a first portion of the second polymer chain including primer reactive side chains;
continuing polymerization with the second backbone monomer to form a second portion of the second polymer chain without primer reactive side chains; and
respectively grafting the cleavable first primer and the un-cleavable second primer to the primer reactive side chains of the first portion of the second polymer chain.

17. The method as defined in claim 16, wherein:
the first functionalized primer set and the second functionalized primer set are generated in separate reaction containers; and
the method further comprises:
mixing the first functionalized primer set and the second functionalized primer set to form a mixture; and
exposing the mixture to the metal surface.

18. The method as defined in claim 17, wherein one of:
the metal surface includes metal nanostructures, and exposing the mixture to the metal surface involves adding the metal nanostructures to the mixture; or
the metal surface includes a transparent metal film positioned on a bottom surface in each of a plurality of depressions of a flow cell, and exposing the mixture to the metal surface involves incubating the mixture in each of the plurality of depressions.

19. A method, comprising:
respectively attaching first and second polymer chains to spatially separate regions of a metal surface, each first polymer chain being attached to the metal surface through a first thiol linkage and each first polymer chain having a first polarity and a first functional group along a backbone of the first polymer chain, and each second polymer chain being attached to the metal surface through a second thiol linkage, and each second polymer chain having a second polarity different from the first polarity and a second functional group along a backbone of the second polymer chain, the second functional group being orthogonal to the first functional group;
respectively grafting an un-cleavable first primer and a cleavable second primer to the first polymer chains through the first functional groups; and
respectively grafting a cleavable first primer and an un-cleavable second primer to the second polymer chains through the second functional groups.

20.-30. (canceled)
